(19) United States
(12) Patent Application Publication
(10) Pub. No.: US 2024/0384304 A1
(43) Pub. Date: Nov. 21, 2024

(54) INHIBITOR OLIGONUCLEOTIDES AND METHODS OF USE THEREOF

(71) Applicant: VOR BIOPHARMA INC., Cambridge, MA (US)

(72) Inventors: Dane Hazelbaker, Cambridge, MA (US); Elizabeth Paik, Cambridge, MA (US); Michael Pettiglio, Cambridge, MA (US); Tirtha Chakraborty, Cambridge, MA (US)

(73) Assignee: VOR BIOPHARMA INC., CAmbridge, MA (US)

(21) Appl. No.: 18/576,181

(22) PCT Filed: Jul. 6, 2022
(86) PCT No.: PCT/US2022/073475

§ 371 Date: Jan. 3, 2024

Related U.S. Application Data
(60) Provisional application No. 63/218,867, filed on Jul. 6, 2021.
(60) Provisional application No. 63/253,477, filed on Oct. 7, 2021.

(51) Int. Cl. C12N 15/90; C12N 9/22; C12N 15/11; C12N 15/113

(52) U.S. Cl. CPC C12N 15/907; C12N 9/22; C12N 15/11; C12N 15/113; C12N 2310/14; C12N 2310/20; C12N 2310/315

(57) ABSTRACT

Provided herein are oligonucleotides comprising a first region that is complementary to a targeting domain of a gRNA and a second region that is complementary to a CRISPR RNA (crRNA) sequence for a CRISPR/Cas nuclease, wherein the oligonucleotide reduces genomic editing at a target sequence complementary to the targeting domain of the gRNA. Also provided herein are methods involving contacting a gRNA, a CRISPR/Cas nuclease, a complex comprising the same, or a cell comprising any thereof with such oligonucleotides.

### RELATED APPLICATIONS

This application claims the benefit under 35 U.S.C. 119(e) of U.S. provisional application No. 63/218,867 filed Jul. 6, 2021, and U.S. provisional application No. 63/253,477 filed on Oct. 7, 2021 each of which are incorporated by reference herein in its entirety.

### REFERENCE TO AN ELECTRONIC SEQUENCE LISTING

The contents of the electronic sequence listing (V029170021WO00-SEQ-CEW.xml; Size: 19,795 bytes; and Date of Creation: Jul. 6, 2022) is herein incorporated by reference in its entirety.

### BACKGROUND

Clustered regulatory Interspaced Short Palindromic Repeats (CRISPR)/Cas systems a provide a platform for targeted gene editing in cells. Despite the versatility of the systems and associated tools for use, there are a number of potential risks associated with genetic modification using CRISPR/Cas systems, such as off-target effects, risk of translocation events, and potential malignancy. These challenges are safety concerns for use of CRISPR/Cas systems in therapeutic applications.

### SUMMARY

Aspects of the present disclosure provide oligonucleotides, comprising a first region that is complementary to a targeting domain of a gRNA and a second region that is complementary to a CRISPR RNA (crRNA) sequence for a CRISPR/Cas nuclease, wherein the oligonucleotide does not occur naturally, wherein the oligonucleotide reduces genomic editing at a target sequence complementary to the targeting domain of the gRNA; and wherein the first region comprises at least 10 nucleotides and the second region comprises at least 10 nucleotides. In some embodiments, the CRISPR/Cas nuclease is Cpf1. In some embodiments, the CRISPR/Cas nuclease is MAD7™, as provided by Inscripta.

In some embodiments, the targeting domain is complementary to a eukaryotic gene. In some embodiments, the oligonucleotide binds to the targeting domain and/or the crRNA sequence and reduces interaction between the targeting domain and/or crRNA sequence and the CRISPR/Cas nuclease. In some embodiments, the oligonucleotide reduces interaction between the gRNA and the CRISPR/Cas nuclease. In some embodiments, the oligonucleotide inhibits formation or maintenance of a ribonucleoprotein (RNP) complex comprising the gRNA and the CRISPR/Cas nuclease. In some embodiments, the oligonucleotide inhibits nuclease activity of a RNP complex comprising the gRNA and the CRISPR/Cas nuclease and/or reduces interaction between the RNP complex and the target sequence in the genome of a cell.

In some embodiments, the first region comprises at least 11, 12, 13, 14, 15, 15, 16, 17, 19, 20, 21, 22, 23, 24, 25, 26, 27, 28, 29, 30, 31, 32, 33, 34, 35, 36, 37, 38, 39, 40, 41, 42, 43, 44, or 45 nucleotides and the second region comprises at least 11, 12, 13, 14, 15, 15, 16, 17, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

In some embodiments, the oligonucleotide comprises one or more nucleotides that comprise a chemical modification. In some embodiments, at least 1, 2, 3, 4, 5, 6, 7, 8, 9, 10, 15, 20, 25, 30, 35, or 40 nucleotides of the oligonucleotide comprise a chemical modification. In some embodiments, at least 10, 20, 50, 75, or 100% of the nucleotides of the oligonucleotide comprise a chemical modification. In some embodiments, the chemical modification is a phosphorothioate linkage. In some embodiments, each nucleotide of the oligonucleotide comprises a phosphorothioate linkage.

In some embodiments, the oligonucleotide is 10-100 nucleotides in length. In some embodiments, the oligonucleotide is 38, 39, 40, 41, 42, 43, 44, or 45 nucleotides in length.

In some embodiments, the oligonucleotide comprises a sequence selected from the group consisting of SEQ ID NOs: 1 and 2. In some embodiments, the second region of the oligonucleotide comprises a sequence of SEQ ID NO: 13.

In some embodiments, the Cpf1 nuclease is derived from Provetella spp. or Francisella spp., Acidaminococcus sp. (AsCpf1), Lachnospiraceae bacterium (LbCpf1), or Eubacterium rectale (ErCas12a). In some embodiments, the Cpf1 nuclease comprises an amino acid sequence with at least 80, 85, 90, 95, 99, or 100% identity to an amino acid sequence selected from the group consisting of SEQ ID NOs: 12, 14, and 15.

Aspects of the present disclosure provide methods of producing a genetically engineered cell, comprising (a) contacting a cell with (i) a first guide RNA (gRNA) and (ii) a CRISPR/Cas nuclease that binds the first gRNA, thus forming a ribonucleoprotein (RNP) complex under conditions suitable for the first gRNA of (i) to form and/or maintain an RNP complex with the CRISPR/Cas nuclease of (ii) and for the RNP complex to bind a first target sequence in the genome of the cell, and (b) contacting the cell with an oligonucleotide, wherein the oligonucleotide reduces genomic editing at the first target sequence.

In some embodiments, the CRISPR/Cas nuclease is Cpf1. In some embodiments, the CRISPR/Cas nuclease is MAD7™, as provided by Inscripta®. In some embodiments, the oligonucleotide comprises a first region that is complementary to a targeting domain of the first gRNA or a portion thereof, and a second region that is complementary to a CRISPR RNA (crRNA) sequence in the first gRNA or a portion thereof.

In some embodiments, the oligonucleotide is an of the oligonucleotides described herein. In some embodiments, the targeting domain of the target gRNA capable of binding a first target sequence, and a CRISPR RNA (crRNA) sequence for the CRISPR/Cas nuclease. In some embodiments, the targeting domain corresponds to the first target sequence adjacent to a protospacer-adjacent motif (PAM) in a genome of the cell. In some embodiments, the contacting of (b) occurs simultaneously or in temporal proximity with the contacting of (a). In some embodiments, the contacting of (b) occurs within 1, 2, 3, 4, 5, 6, 7, 8, 9, 10, 11, 12, 18, or 24 hours after the contacting of (a).

In some embodiments, the method further comprises (c) contacting the cell with (iii) a second gRNA comprising a second targeting domain capable of binding a second target sequence and a crRNA sequence for a CRISPR/Cas nuclease; wherein the second targeting domain and second target sequence are different than the targeting domain of the first gRNA and the first target sequence. In some embodiments, (c) further comprises contacting the cell with (iv) a CRISPR/Cas nuclease that binds the second gRNA, thus forming a ribonucleoprotein (RNP) complex under conditions suitable for the second gRNA of (iii) to form and/or maintain an RNP complex with the CRISPR/Cas nuclease of (iv) and for the RNP complex to bind a second target sequence in the genome of the cell.

In some embodiments, the CRISPR/Cas nuclease of (iv) does not comprise a Cpf1 nuclease. In some embodiments, the CRISPR/Cas nuclease of (iv) is a Cas9 nuclease. In some embodiments, the CRISPR/Cas nuclease of (iv) is a Cas9 nuclease. In some embodiments, the CRISPR/Cas nuclease of (iv) comprises a Cpf1 nuclease.

In some embodiments, the contacting of (c) occurs simultaneously or in temporal proximity with the contacting of (b). In some embodiments, the contacting of (c) occurs within 1, 2, 3, 4, 5, 6, 7, 8, 9, 10, 11, 12, 18, or 24 hours after the contacting of (b). In some embodiments, the method further comprises (d) contacting the cell with a second oligonucleotide, wherein the second oligonucleotide reduces genomic editing at the second target sequence. In some embodiments, the contacting of (d) and the contacting of (c) occur simultaneously or in temporal proximity to one another. In some embodiments, the contacting of (d) occurs within 1, 2, 3, 4, 5, 6, 7, 8, 9, 10, 11, 12, 18, or 24 hours after the contacting of (c). In some embodiments, the second oligonucleotide is any of the oligonucleotides described herein.

In some embodiments, the oligonucleotide of (b) does not substantially bind the second gRNA of (iii) and/or a RNP complex of the second gRNA of (iii) and the CRISPR/Cas nuclease of (iv). In some embodiments, the oligonucleotide of (b) does not substantially inhibit formation or maintenance of the RNP complex comprising the second gRNA of (iii) and the CRISPR/Cas nuclease of (ii) or the CRISPR/Cas nuclease of (iv). In some embodiments, the oligonucleotide of (b) does not substantially inhibit the ability of the RNP complex comprising the second gRNA of (iii) and the CRISPR/Cas nuclease of (ii) or the CRISPR/Cas nuclease of (iv) to bind the second target sequence in the genome of the cell. In some embodiments, the oligonucleotide of (b) does not substantially inhibit nuclease activity of the RNP complex comprising the second gRNA of (iii) and the CRISPR/Cas nuclease of (ii) or the CRISPR/Cas nuclease of (iv) to bind the second target sequence in the genome of the cell. In some embodiments, the second oligonucleotide of (d) does not substantially bind the gRNA of (i) and/or the RNP complex comprising the first gRNA of (i) and the CRISPR/Cas nuclease of (ii). In some embodiments, the second oligonucleotide of (d) does not substantially inhibit formation or maintenance of the RNP complex comprising the first gRNA of (i) and the CRISPR/Cas nuclease of (ii). In some embodiments, the second oligonucleotide of (d) does not substantially inhibit the ability of the RNP complex comprising the first gRNA of (i) and the CRISPR/Cas nuclease of (ii) to bind the first target sequence in the genome.

In some embodiments, the second oligonucleotide of (d) does not substantially inhibit nuclease activity of the RNP complex comprising the first gRNA of (i) and the CRISPR/Cas nuclease of (ii) to bind the first target sequence in the genome of the cell.

In some embodiments, the cell is a hematopoietic cell. In some embodiments, the hematopoietic cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is selected from the group consisting of an embryonic stem cell (ESC), an induced pluripotent stem cell (iPSC), a mesenchymal stem cell, and a tissue-specific stem cell.

In some embodiments, the contacting of (a) comprises introducing (i) and (ii) into the cell in the form of a pre-formed ribonucleoprotein (RNP) complex. In some embodiments, the contacting of (c) comprises introducing the second gRNA of (iii) and the CRISPR/Cas nuclease of (iv) into the cell in the form of a pre-formed ribonucleoprotein (RNP) complex. In some embodiments, the contacting of (a) comprises introducing (i) and/or (ii) into the cell in the form of a nucleic acid encoding the first gRNA of (i), and/or the CRISPR/Cas nuclease of (ii); and/or the contacting of (b) comprises introducing the oligonucleotide into the cell in the form of a nucleic acid encoding the oligonucleotide. In some embodiments, the nucleic acid encoding the first gRNA of (i), and/or the CRISPR/Cas nuclease of (ii), and/or the oligonucleotide is an RNA, preferably an mRNA or an mRNA analog. In some embodiments, the contacting of (c) comprises introducing the second gRNA of (iii) and/or the CRISPR/Cas nuclease of (iv) into the cell in the form of a nucleic acid encoding the second gRNA of (iii) and/or the second CRISPR/Cas nuclease of (iv); and/or the contacting of (d) comprises introducing the second oligonucleotide into the cell in the form of a nucleic acid encoding the second oligonucleotide.

In some embodiments, the nucleic acid encoding the second gRNA of (iii), the second CRISPR/Cas nuclease of (iv), and/or the second oligonucleotide is an RNA, preferably an mRNA or an mRNA analog.

In some embodiments, the ribonucleoprotein complex is introduced into the cell via electroporation.

Aspects of the present disclosure provide genetically engineered cells, or descendants thereof, produced by any of the methods described herein. Aspects of the present disclosure provide cell populations, comprising any of the genetically engineered cells, or a descendant thereof, described herein.

Aspects of the present disclosure provide pharmaceutical compositions comprising the cell, or a descendant thereof, or any of the cell populations described herein.

Aspects of the present disclosure provide ribonucleoprotein particles (RNP) comprising a CRISPR/Cas nuclease, a first gRNA, and any of the oligonucleotides described herein. In some embodiments, the CRISPR/Cas nuclease is Cpf1. In some embodiments, the CRISPR/Cas nuclease is MAD7™.

Aspects of the present disclosure provide a system comprising a CRISPR/Cas nuclease, a first gRNA, any of the oligonucleotides described herein, and a second gRNA. In some embodiments, the CRISPR/Cas nuclease is Cpf1. In some embodiments, the CRISPR/Cas nuclease is MAD7™. In some embodiments, the system further comprises a second CRISPR/Cas nuclease.

Aspects of the present disclosure provide methods comprising administering to a subject in need thereof any of the cells, or descendants thereof, cell populations, or the pharmaceutical compositions described herein. In some embodiments, the cell or descendant thereof or the cells of the cell population comprise a modification in a first gene relative to a wild-type counterpart cell. In some embodiments, the cell or descendant thereof or the cells of the cell population comprise a modification to a second gene relative to a wild-type cell of the same type. In some embodiments, the method further comprises administering to the subject a therapeutically effective amount of at least one agent that targets a product encoded by the first gene or a wildtype copy thereof, wherein the agent comprises an antigen binding fragment that binds the product encoded by the first gene or a wildtype copy thereof.

In some embodiments, administration of the at least one agent targeting the product encoded by the first gene or a wildtype copy thereof occurs simultaneously or in temporal proximity with administration of any of the cells, or descendants thereof, cell populations, or pharmaceutical compositions described herein.

In some embodiments, administration of the at least one agent targeting the product encoded by the first gene or a wildtype copy thereof occurs after administration of any of the cells, or descendants thereof, cell populations, or pharmaceutical compositions described herein.

In some embodiments, administration of the at least one agent targeting the product encoded by the first gene or a wildtype copy thereof occurs before administration of any of the cells, or descendants thereof, cell populations, or pharmaceutical compositions described herein. In some embodiments, the method further comprises administering to the subject a therapeutically effective amount of at least one agent that targets a product encoded by the second gene or a wildtype copy thereof, wherein the agent comprises an antigen binding fragment that binds the product encoded by the second gene or a wildtype copy thereof. In some embodiments administration of the at least one agent targeting the product encoded by the second gene or a wildtype copy thereof occurs simultaneously or in temporal proximity with administration of any of the cells, or descendants thereof, cell populations, or pharmaceutical compositions described herein.

In some embodiments, administration of the at least one agent targeting the product encoded by the second gene or a wildtype copy thereof occurs after administration of any of the cells, or descendants thereof, cell populations, or pharmaceutical compositions described herein.

In some embodiments, administration of the at least one agent targeting the product encoded by the second gene or a wildtype copy thereof occurs before administration of any of the cells, or descendants thereof, cell populations, or pharmaceutical compositions described herein.

In some embodiments, administration of the at least one agent targeting the product encoded by the second gene or a wildtype copy thereof occurs simultaneously or in temporal proximity with administration of the at least one agent targeting the product encoded by the first gene or a wildtype copy thereof. In some embodiments, administration of the at least one agent targeting the product encoded by the second gene or a wildtype copy thereof occurs after administration of the at least one agent targeting the product encoded by the first gene or a wildtype copy thereof. In some embodiments, administration of the at least one agent targeting the product encoded by the second gene or a wildtype copy thereof occurs before administration of the at least one agent targeting the product encoded by the first gene or a wildtype copy thereof.

In some embodiments, the agent that targets a product encoded by the first gene or a wildtype copy thereof and/or the agent that targets a product encoded by the second gene or a wildtype copy thereof is cytotoxic agent. In some embodiments, the cytotoxic agent is an antibody-drug conjugate or an immune effector cell expressing a chimeric antigen receptor (CAR). In some embodiments, the subject has a disease associated with cells expressing the modified gene or a wildtype copy thereof. In some embodiments, the subject has a cancer associated with cancer stem cells. In some embodiments, the subject has a hematopoietic malignancy. In some embodiments, the subject has an autoimmune disease.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Examples, and the Claims.

### BRIEF DESCRIPTION OF THE FIGURES

FIG. 1 shows an exemplary experimental workflow. CD34+ cells are thawed and 48 hours later are electroporated with ribonucleoproteins (RNPs), gRNAs targeting a target site, with or without co-electroporation of inhibitor oligonucleotides. A sample of cells (approximately 150,000 cells) are harvested and assessed for viability at 4 hours, 24 hours, 36 hours, and 48 hours post-electroporation (post-EP). After 48 hours post-electroporation, DNA is extracted from the cells, and genomic editing is assessed by DNA sequencing.

FIGS. 2A-2B show the effect of electroporation with ribonulceoproteins (RNPs) comprising a Cpf1 nuclease and gRNAs targeting genes encoding CCR5 or CD19 on cell viability. FIG. 2A shows a graph of cell growth at the indicated time points after cells were electroporated in the absence or in the presence of inhibitor oligonucleotides (denoted by “+Inh” or “−Inh” in legend). FIG. 2B shows of the percent cell viability at the indicated time points after cells were electroporated in the absence or in the presence of inhibitor oligonucleotides (denoted by “+Inh” or “−Inh” in legend). “Culture” refers to control cells that were not electroporated with the RNPs.

FIGS. 3A and 3B show graphs of on-target editing assessed by Next Generation Sequencing (NGS) at the indicated time points after cells were electroporated with RNPs comprising a Cpf1 nuclease from Acidaminococcus sp. BV3L6 (AsCpf1) and gRNAs targeting genes encoding CCR5 (FIG. 3A) or CD19 (FIG. 3B)), with or without co-electroporation of inhibitor oligonucleotides (denoted by “+Inh” or “−Inh” in legend).

FIG. 4 shows an exemplary experimental workflow. CD34+ cells are thawed and 2 days later a population of cells are electroporated with ribonucleoprotein (RNP) complexes containing gRNAs targeting a target site (e.g., CCR5, CD19) and Cpf1 and inhibitor oligonucleotides (“I0”). The remaining cells are bulk edited by electroporated with ribonucleoprotein (RNP) complexes containing gRNAs targeting a target site and Cpf1. The bulked edited cells are then electroporated with inhibitor oligonucleotides at 8 hours (“I8”) or 36 hours (“I36”) following the first electroporation. Cells are harvested 48 hours following the first electroporation, DNA is extracted from the cells, and genomic editing is assessed by Next Generation Sequencing (NGS) analysis.

FIGS. 5A and 5B show graphs of cell viability at the indicated time points after cells were electroporated with RNPs comprising a Cpf1 nuclease and gRNAs targeting exemplary target sites. FIG. 5A shows percent cell viability for cells electroporated with a Cpf1 nuclease and gRNAs targeting CCR5. FIG. 5B shows percent cell viability for cells electroporated with a Cpf1 nuclease and gRNAs targeting CD19. At time 0 hours (I0), 8 hours (I8), or 36 hours (I36) post-RNP electroporation, the indicated cells were electroporated with inhibitor oligonucleotides specific for the target sites. Control cells that were not electroporated with RNPs are indicated as “NoEP” and were also electroporated with the inhibitor oligonucleotides at a time corresponding to 8 hours (I8) or 36 hours (I36).

FIG. 6 shows a graph of on-target editing as assessed by Next Generation Sequencing (NGS) after cells were electroporated with RNPs comprising a Cpf1 nuclease from Acidaminococcus sp. BV3L6 (AsCpf1) and gRNAs targeting exemplary the genes encoding CCR5 or CD19. Cells were either not contacted with the oligonucleotide inhibitors or electroporated with the oligonucleotide inhibitors specific for the target site at time 0 hours (I0), 8 hours (I8), or 36 hours (I36) post-RNP electroporation.

FIG. 7 shows an exemplary experimental workflow. CD34+ cells are thawed and 2 days later a population of cells is electroporated with ribonucleoprotein (RNP) complexes containing gRNAs targeting a target site (e.g., CCR5, CD19, CD5) and AsCpf1 or SpCas9 nuclease with or without inhibitor oligonucleotides. As indicated, the inhibitor oligonucleotides were directed to target site or a different target site. Cells are harvested 4, 8, 24, and 48 hours following the electroporation, DNA is extracted from the cells, and genomic editing is assessed by Next Generation Sequencing (NGS) analysis.

FIGS. 8A-8B show the effect of electroporation with RNPs comprising a Cpf1 nuclease (unlabeled) or Cas9 nuclease (“Cas9”) and gRNAs targeting genes encoding CCR5, CD19, or CCR5 on cell viability. FIG. 8A shows a graph of percent cell viability at the indicated time points of cells that were electroporated in the absence or in the presence of inhibitor oligonucleotides specific for the target site or inhibitor oligonucleotides specific for a different target site (oligonucleotide specificity indicated in paratheses). FIG. 8B shows a graph of cell growth at the indicated time points of cells that were electroporated in the absence or in the presence of inhibitor oligonucleotides specific for the target site or inhibitor oligonucleotides specific for a different target site (oligonucleotide specificity indicated in paratheses). Control cells that were not electroporated with RNPs are indicated as “No EP.”

FIGS. 9A and 9B show graphs of on-target editing as assessed by Next Generation Sequencing (NGS) at the indicated time points after cells were electroporated with RNPs comprising the Cpf1 nuclease from Acidaminococcus sp. BV3L6 and gRNAs targeting the genes encoding CCR5 (FIG. 9A) or CD19 (FIG. 9B). Cells were co-electroporated without or with (“+Inh”) inhibitor oligonucleotides specific for the target site or inhibitor oligonucleotides specific for a different target site (oligonucleotide specificity indicated in parentheses).

FIG. 10 shows a graph of on-target editing as assessed by Next Generation Sequencing (NGS) at the indicated time points after cells were electroporated with RNPs comprising a Cas9 from Streptococcus pyogenes (SpCas9) nuclease and gRNAs targeting the gene encoding CD5. Cells were electroporated without or with (“+Inh”) Cpf1 nuclease inhibitor oligonucleotides specific for the target site or inhibitor oligonucleotides specific for a different target site (e.g., CD19; oligonucleotide specificity indicated in parentheses).

FIGS. 11A and 11B show graphs of on-target editing efficiency as assessed by Next Generation Sequencing (NGS). FIG. 11A shows on-target editing efficiency of cells that were simultaneously electroporated with RNPs comprising a Cpf1 (AsCpf1) nuclease and gRNAs targeting the gene encoding CD19 (AsCpf1 CD19) and RNPs comprising a Cas9 nuclease from Streptococcus pyogenes (SpCas9) and gRNAs targeting the gene encoding CD5 (SpCas9 CD5) (“Si CD19+CD5”); or sequentially electroporated with RNPs comprising a Cpf1 (AsCpf1) nuclease and gRNAs targeting the gene encoding CD19 (AsCpf1 CD19) followed by electroporation with RNPs comprising a Cas9 nuclease from Streptococcus pyogenes (SpCas9) and gRNAs targeting the gene encoding CD5 (SpCas9 CD5), in the absence of a Cpf1 nuclease inhibitor oligonucleotide (“Se CD19>CD5”) or in the presence of a Cpf1 nuclease inhibitor oligonucleotide (“Se CD19>CD5+Inh”). For each editing strategy, the left column corresponds to AsCpf1 CD19 editing and the right column corresponds to SpCas9 CD5 editing. FIG. 11B shows on-target editing efficiency of cells that were simultaneously electroporated with RNPs comprising a Cpf1 (AsCpf1) nuclease and gRNAs targeting the gene encoding CCR5 (AsCpf1 CCR5) and RNPs comprising a Cas9 nuclease from Streptococcus pyogenes (SpCas9) and gRNAs targeting the gene encoding CD5 (SpCas9 CD5) (“Si CCR5+CD5”); or sequentially electroporated with RNPs comprising a Cpf1 (AsCpf1) nuclease and gRNAs targeting the gene encoding CCR5 (AsCpf1 CCR5) followed by electroporation with RNPs comprising a Cas9 nuclease from Streptococcus pyogenes (SpCas9) and gRNAs targeting the gene encoding CD5 (SpCas9 CD5), in the absence of a Cpf1 nuclease inhibitor oligonucleotide (“Se CCR5>CD5”) or in the presence of a Cpf1 nuclease inhibitor oligonucleotide (“Se CCR5>CD5+Inh”). For each editing strategy, the left column corresponds to AsCpf1 CCR5 editing and the right column corresponds to SpCas9 CD5 editing.

FIGS. 12A-12D show graphs of balanced genomic translocation (on-on translocation) by amplicon sequencing using the rhAmpSeq sequencing system. FIG. 12A shows the percentage of balanced translocation events detected in cells that were simultaneously electroporated with RNPs comprising a Cpf1 (AsCpf1) nuclease and gRNAs targeting the gene encoding CD19 (AsCpf1 CD19) and RNPs comprising a Cas9 nuclease from Streptococcus pyogenes (SpCas9) and gRNAs targeting the gene encoding CD5 (SpCas9 CD5) (“Si CD19+CD5”); or sequentially electroporated with RNPs comprising a Cpf1 (AsCpf1) nuclease and gRNAs targeting the gene encoding CD19 (AsCpf1 CD19) followed by electroporation with RNPs comprising a Cas9 nuclease from Streptococcus pyogenes (SpCas9) and gRNAs targeting the gene encoding CD5 (SpCas9 CD5), in the absence of a Cpf1 nuclease inhibitor oligonucleotide (“Se CD19>CD5”) or in the presence of a Cpf1 nuclease inhibitor oligonucleotide (“Se CD19>CD5+Inh”). FIG. 12B shows the total balanced, total ascentric, and total dicentric translocation events detected using a quantification window size of 25 bp for the sequencing data shown in FIG. 12A as determined by UDITAS analysis. FIG. 12C shows the percentage of balanced translocation events detected in cells that were simultaneously electroporated with RNPs comprising a Cpf1 (AsCpf1) nuclease and gRNAs targeting the gene encoding CCR5 (AsCpf1 CCR5) and RNPs comprising a Cas9 nuclease from Streptococcus pyogenes (SpCas9) and gRNAs targeting the gene encoding CD5 (SpCas9 CD5) (“Si CCR5+CD5”); or sequentially electroporated with RNPs comprising a Cpf1 (AsCpf1) nuclease and gRNAs targeting the gene encoding CCR5 (AsCpf1 CCR5) followed by electroporation with RNPs comprising a Cas9 nuclease from Streptococcus pyogenes (SpCas9) and gRNAs targeting the gene encoding CD5 (SpCas9 CD5), in the absence of a Cpf1 nuclease inhibitor oligonucleotide (“Se CCR5>CD5”) or in the presence of a Cpf1 nuclease inhibitor oligonucleotide (“Se CCR5>CD5+Inh”). FIG. 12D shows the total balanced, total ascentric, and total dicentric translocation events detected using a quantification window size of 25 bp for the sequencing data shown in FIG. 12C as determined by rhAmpSeq analysis.

FIGS. 13A-13C show the effect of crRNA:AsCpf1:Inh titration on editing outcomes at the CCR5 and CD19 loci and cell viability. FIG. 13A shows an exemplary dosing approach for titration of the molar ratios of cRNA:AsCpf1:Inh during editing procedures. FIG. 13B shows editing frequency determined via Inference of CRISPR Editing (“ICE”) analysis (left panel), cell counts (middle panel), and cell viability (right panel) in cell populations electroporated with RNPs targeting a gene encoding CCR5. FIG. 13C shows editing frequency determined via Inference of CRISPR Editing (“ICE”) analysis (left panel), cell counts (middle panel), and cell viability (right panel) in cell populations treated with RNPs targeting a gene encoding CD19. Numerical values listed on the X-axis correspond to the dosage in micrograms of crRNA, AsCpf1, and inhibitor oligonucleotides, respectively.

FIG. 14 shows an exemplary experimental workflow for the experiments shown in FIGS. 11A, 11B, 12A-12D, 15A, and 15B. CD34+ donor cells are thawed, cultured, and on day 2 a population of cells are electroporated with a first ribonucleoprotein (RNP) complex containing gRNAs targeting a target site (e.g., CCR5, CD19) and AsCpf1 or both AsCpf1 and SpCas9. After 30 hours, cells are electroporated with a second RNP complex containing gRNAs targeting a target site (e.g., CCR5, CD19) and SpCas9 in the absence or in the presence of an AsCpf1 inhibitor oligonucleotide. Cells are harvested 24 hours following the second electroporation, DNA is extracted from the cells, and genomic editing is assessed by Next Generation Sequencing (NGS) analysis.

FIGS. 15A-15B show the effect of simultaneous (“Si”) and sequential (“Se”) electroporation with RNPs targeting a target site (e.g., CCR5, CD19, CD5) in the absence or the presence of inhibitor oligonucleotides on cell viability. FIG. 15A shows cell count analysis taken at the indicated time points in cells that were either simultaneously electroporated with RNPs targeting the genes encoding CD19 and CD5 (“Si CD19+CD5”), sequentially electroporated (“EP1) with a first RNPs targeting the gene encoding CD19 and a second RNP targeting CD5 in the absence (“Se CD19>CD5”) or in the presence (“Se CD19>CD5+In”) of inhibitor oligonucleotides, or sequentially electroporated with a first RNP targeting CCR5 and second RNP targeting CD5 in the absence (“Se CCR5>CD5”) or in the presence (“Se CCR5>CD5 In”) of inhibitor oligonucleotides. FIG. 15B shows percent viability cells at the indicated time points. The cells were either simultaneously electroporated with RNPs targeting the genes encoding CD19 and CD5 (“Si CD19+CD5”), sequentially electroporated (“EP1) with a first RNP targeting the gene encoding CD19 and a second RNP targeting CD5 in the absence (“Se CD19>CD5”) or in the presence (“Se CD19>CD5+In”) of inhibitor oligonucleotides, or sequentially electroporated with a first RNP targeting CCR5 and second RNP targeting CD5 with in the absence (“Se CCR5>CD5”) or in the presence (“Se CCR5>CD5 In”) of inhibitor oligonucleotides. Control cells that were not subjected to electroporation are indicated as “No EP”.

FIG. 16 shows a graph comparing balanced genomic translocation (on-on translocation) determined by amplicon sequencing using the rhAmpSeq sequencing system for current multiplex editing methods. Cells were subjected to either simultaneous electroporation with a low concentration of RNPs comprising a Cas9 nuclease from Streptococcus pyogenes (SpCas9) and gRNAs targeting the genes encoding CD33 and CD5 (“Viivs006_SiLo_Cas9(1)+Cas9(2)”), simultaneous electroporation with a high concentration of RNPs comprising a Cas9 nuclease from Streptococcus pyogenes (SpCas9) and gRNAs targeting the genes encoding CD33 and CD5 (“Viivs006_SiHi_Cas9(1)+Cas9(2)”), simultaneous electroporation with a high concentration of RNPs comprising a Cas9 nuclease from Streptococcus pyogenes (SpCas9) and gRNAs targeting the genes encoding CD33 and CD5 (“Viivs023_SiHiCas9(3)+Cas9(4)”), simultaneous electroporation with RNPs comprising a Cas9 nuclease from Streptococcus pyogenes (SpCas9) and gRNAs targeting the genes encoding CD33 and CD5 (“Viivs006_SiHi_Cas9(1)+Cas9(2)”), simultaneous electroporation with RNP comprising Cpf1 (AsCpf1) and crRNA targeting the gene encoding CD19 and RNP comprising Cas9 nuclease from Streptococcus pyogenes (SpCas9) and gRNA targeting the gene encoding CD5 (“Si AsCpf1(A)+ SpCas9(C)”), simultaneous electroporation with RNP comprising Cpf1 (AsCpf1) and crRNA targeting the gene encoding CCR5 and RNP comprising Cas9 nuclease from Streptococcus pyogenes (SpCas9) and gRNAs targeting the gene encoding CD5 (“Si_AsCpf1(B)+SpCas9(C)”), sequential electroporation with a first RNP comprising Cas9 nuclease from Streptococcus pyogenes (SpCas9) and gRNA targeting the gene encoding CD33 and a second RNP comprising Cas9 nuclease from Streptococcus pyogenes (SpCas9) and gRNA targeting the gene encoding CD5 (“Viivs006_Se_Cas9(1)>Cas9(2)”), sequential electroporation with a first RNP comprising Cas9 nuclease from Streptococcus pyogenes (SpCas9) and gRNA targeting the gene encoding CD5 and a second RNP comprising Cas9 nuclease from Streptococcus pyogenes (SpCas9) and gRNA targeting the gene encoding CD33 (“Viivs006_Se_Cas9(2)>Cas9(1)”), sequential electroporation with a first RNP comprising Cas9 nuclease from Streptococcus pyogenes (SpCas9) and gRNA targeting the gene encoding CD33 and a second RNP comprising Cas9 nuclease from Streptococcus pyogenes (SpCas9) and gRNA targeting the gene encoding CD5 (“Viivs006_Se_Cas9(1)>Cas9(2)”), sequential electroporation with a first RNP comprising Cas9 nuclease from Streptococcus pyogenes (SpCas9) and gRNA targeting the gene encoding CD5 and a second RNP comprising Cas9 nuclease from Streptococcus pyogenes (SpCas9) and gRNA targeting the gene encoding CD33 (“Viivs007_Ase_Cas9(1)>Cas9(2)_90”), sequential electroporation with a first RNP comprising Cas9 nuclease from Streptococcus pyogenes (SpCas9) and gRNA targeting the gene encoding CD5 and a second RNP comprising Cas9 nuclease from Streptococcus pyogenes (SpCas9) and gRNA targeting the gene encoding CD33 (“Viivs007_Ase_Cas9(1)>Cas9(2)_112”), sequential electroporation with a first RNP comprising Cas9 nuclease from Streptococcus pyogenes (SpCas9) and gRNA targeting the gene encoding CD5 and a second RNP comprising Cas9 nuclease from Streptococcus pyogenes (SpCas9) and gRNA targeting the gene encoding CD33 (“Viivs007_Bse_Cas9(1)>Cas9(2)_112”), sequential electroporation with a first RNP comprising Cas9 nuclease from Streptococcus pyogenes (SpCas9) and gRNA targeting the gene encoding CD33 and a second RNP comprising Cas9 nuclease from Streptococcus pyogenes (SpCas9) and gRNA targeting the gene encoding CLL-1 (“Viivs023_Cas9(3)>Cas9(4)”), sequential electroporation with a first RNP comprising Cas9 nuclease from Streptococcus pyogenes (SpCas9) and gRNA targeting the gene encoding CLL-1 and a second RNP comprising Cas9 nuclease from Streptococcus pyogenes (SpCas9) and gRNA targeting the gene encoding CD33 (“Viivs023_Cas9(4)>Cas9(3)”), sequential electroporation with a first RNP comprising Cpf1 (AsCpf1) and crRNA targeting the gene encoding CD19 and a second RNP comprising Cas9 nuclease from Streptococcus pyogenes (SpCas9) and gRNA targeting the gene encoding CD5 (“Se_AsCpf1(A)>SpCas9(C)”), sequential electroporation with a first RNP comprising Cpf1 (AsCpf1) and crRNA targeting the gene encoding CD19 and a second RNP comprising Cas9 nuclease from Streptococcus pyogenes (SpCas9) and gRNA targeting the gene encoding CD5 in addition to oligonucleotide inhibitor specific to the Cpf1 and crRNA targeting the gene encoding CD19 RNP (“Se_AsCpf1(A)>SpCas9(C)+Inh”), sequential electroporation with a first RNP comprising Cpf1 (AsCpf1) crRNA targeting the gene encoding CCR5 and a second RNP comprising Cas9 nuclease from Streptococcus pyogenes (SpCas9) and gRNA targeting the gene encoding CD5 (“Se_AsCpf1(B)>SpCas9(C)”), or sequential electroporation with a first RNP comprising Cpf1 (AsCpf1) and Cas9 nuclease from Streptococcus pyogenes (SpCas9) and gRNA targeting the gene encoding CCR5 and a second RNP comprising Cpf1 (AsCpf1) and Cas9 nuclease from Streptococcus pyogenes (SpCas9) and gRNA targeting the gene encoding CD5 in addition to oligonucleotide inhibitor specific to the Cpf1 and crRNA targeting the gene encoding CCR5 RNP (“Se_AsCpf1(B)>SpCas9(C)+Inh”).

### DETAILED DESCRIPTION

Use of CRISPR/Cas systems to effect genetic modifications presents a versatile and adaptable platform, however, there are a number of potential risks associated with CRISPR/Cas use in therapeutic applications, such as off-target effects, risk of translocation events, and potential malignancy. To minimize or reduce potential adverse effects, mechanisms of regulating the activity of the CRISPR/Cas system in the cell, for example to induce or terminate its activity and thus DNA cleavage, are desired. Despite existing methods of controlling CRISPR/Cas activity in a cell including anti-CRISPR proteins adapted from bacteriophage, small molecule inhibitors, and oligonucleotide inhibitors, each of these control systems present challenges such as delivery of the inhibitor, incomplete inactivation of the CRISPR/Cas system, and slow kinetics. See, e.g., Harrington et al. Cell (2017) 170(6): 1224-1233; Pawluk et al. Nat. Microbiol. (2016) 1(8): 16085; Pawluk et al. mBio (2018) 9(6): e023121-18; Shin et al. Cell (2017) 170(6): 1224-1233.e15; Kundert et al. Nat. Commun. (2019) 10:2127; Maji et al. Cell (2019) 177(4): 1067-1079; Barkau et al. Nucleic Acid Ther. (2019) 29(3): 136-147; Li et al. Cell Rep. (2018) 25(12): 3262-3272.

Aspects of the present disclosure provide oligonucleotides that reduce genomic editing by a CRISPR/Cas nuclease (e.g., Cpf1 nuclease) at a target sequence (e.g., in the genome of a cell) complementary to a targeting domain of a gRNA. The oligonucleotides described herein are effective in preventing, reducing, and/or terminating genomic editing at a target sequence in a sequence-specific and nuclease-specific manner. The present disclosure is directed, at least in part, to the development of oligonucleotides comprising a first region that is complementary to a targeting domain of a gRNA (or portion thereof) and a second region that is complementary to a CRISPR RNA (crRNA) sequence for a CRISPR/Cas nuclease (e.g., Cpf1 nuclease). Also described herein are methods of producing genetically engineered cells involving contacting a cell with a gRNA and a CRISPR/Cas nuclease (e.g., Cpf1 nuclease) such that the gRNA and nuclease form a ribonucleoprotein (RNP) complex that binds to a target sequence in the genome of the cell, and contacting the cell with any of the oligonucleotides described herein. The cells may be further contacted with a second gRNA, and optionally an additional CRISPR/Cas nuclease, to effect a genetic modification (e.g., a mutation) in the genome of a cell. Also provided herein are systems comprising a gRNA, a CRISPR/Cas nuclease (e.g., Cpf1 nuclease), and any of the oligonucleotides described herein. Also provided herein are methods involving administering any of the genetically engineered cells genetic modification (e.g., a mutation), or descendants thereof, produced by the methods described herein to a subject.

The term “mutation,” as used herein, refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence. In some embodiments provided herein, a mutation in a gene (e.g., a target gene) results in a loss of expression of a protein encoded by the target gene in a cell harboring the mutation. In some embodiments, a mutation in a gene (e.g., a target gene) results in the expression of a variant form of a protein that is encoded by the target gene.

Some aspects of this disclosure provide compositions and methods for generating the genetically engineered cells described herein, e.g., genetically engineered cells comprising a modification in their genome, such as a modification that results in a loss of expression or regulation of a protein, or expression of a variant form of a protein. Such compositions and methods provided herein include, without limitation, suitable strategies and approaches for genetically engineering cells, e.g., by using RNA-guided nucleases, such as CRISPR/Cas nucleases, and suitable RNAs able to bind such RNA-guided nucleases and target them to a suitable target site within the genome of a cell to effect a genomic modification.

In some embodiments, a genetically engineered cell described herein is generated via genome editing technology, which includes any technology capable of introducing targeted changes, also referred to as “edits,” into the genome of a cell. The oligonucleotides described herein are effective in preventing, reducing, and/or terminating genomic editing, for example at a desired time.

### Oligonucleotides

Aspects of the present disclosure relate to oligonucleotides that are not naturally occurring and are capable of reducing genomic editing at a target sequence complementary to the targeting domain of a guide RNA (gRNA) of a CRISPR/Cas nuclease (e.g., Cpf1 (Cas12a)) CRISPR/Cas system. As used herein, the term “oligonucleotide” refers to a non-naturally occurring nucleic acid molecule comprising between 10 and 100 nucleotides. In some embodiments, the oligonucleotide comprises one or more DNA nucleotides. In some embodiments, the oligonucleotide comprises one or more RNA nucleotides. In some embodiments, the oligonucleotide comprises a mixture of DNA and RNA nucleotides.

Without wishing to be bound by any particular theory, the oligonucleotides described herein are capable of preventing, reducing, and/or eliminating genomic editing at a target sequence complementary to the targeting domain of a gRNA. In some embodiments, the oligonucleotide reduces interaction between a gRNA and a CRISPR/Cas nuclease (e.g., Cpf1 nuclease), thereby reducing genomic editing at a target sequence. In some embodiments, the oligonucleotide binds to the targeting domain a gRNA and reduces interaction between the targeting domain and the CRISPR/Cas nuclease (e.g., Cpf1 nuclease). In some embodiments, the oligonucleotide binds to the targeting domain a CRISPR RNA (crRNA) sequence for a Cpf1 nuclease and reduces interaction between the crRNA and the Cpf1 nuclease. In some embodiments, the targeting domain is complementary to a eukaryotic gene.

As described herein, a gRNA and a CRISPR/Cas nuclease (e.g., Cpf1) forms a ribonucleoprotein complex comprising the gRNA and the CRISPR/Cas nuclease, thus forming a CRISPR system, and allows the RNP complex to bind a target site sequence in the genome of the cell, resulting in genomic editing at the target site sequence. In some embodiments, the oligonucleotide inhibits the formation of a RNP complex comprising a gRNA and a CRISPR/Cas nuclease (e.g., Cpf1 nuclease). In some embodiments, the oligonucleotide inhibits the maintenance of a RNP complex comprising a gRNA and a CRISPR/Cas nuclease (e.g., Cpf1 nuclease). In some embodiments, the oligonucleotide inhibits nuclease activity of the RNP complex comprising a gRNA and a CRISPR/Cas nuclease (e.g., Cpf1 nuclease). In some embodiments, the oligonucleotide reduces interaction between the RNP complex and the target site sequence in the genome of the cell.

Without being bound by any of the above exemplary modalities, an oligonucleotide described herein may inactivate a CRISPR/Cas system with respect to a particular gRNA or target sequence. The oligonucleotides provided herein may thus provide temporal and/or spatial control over the activity a CRISPR/Cas system. Such control may decrease off-target activity of the CRISPR/Cas system or any toxic effects on a biological system being altered (e.g., on a subject being treated or a cell or plurality of cells being genetically engineered), for example, by inactivating the CRISPR/Cas system after sufficient effect (e.g., genetic modification or targeting of non-genetic modification effect to a target sequence) has been achieved. Through the use of multiple oligonucleotides, multiple (e.g., sequential) CRISPR/Cas system-mediated alterations to a cell may be controlled, e.g., using a first oligonucleotide specific for a first gRNA or first target sequence and a second oligonucleotide specific for a second gRNA or second target sequence.

The oligonucleotides described herein comprise a plurality of regions, wherein each region is capable of binding to (e.g., complementary to) a sequence on another nucleic acid molecule, for example, a portion of a gRNA. In some embodiments, the oligonucleotide comprises a first region that is complementary to a targeting domain of a gRNA (or portion thereof) and a second region that is complementary to a CRISPR RNA (crRNA) sequence (or a portion thereof) for a Cpf1 nuclease. An oligonucleotide comprising a first region that is complementary to a targeting domain of a gRNA and a second region that is complementary to a crRNA sequence of the gRNA for a Cpf1 nuclease is referred to herein as being “specific” to the particular gRNA. An oligonucleotide comprising a first region that is complementary to a targeting domain of a gRNA, where the targeting domain is complementary to a target sequence (e.g., in the genome of a cell), may also be referred to as being “specific” to the target sequence.

As described herein, the nucleic acid sequence(s) of one or more regions of the oligonucleotides may be naturally occurring sequences, however the oligonucleotide is not naturally occurring. For example, the combination of regions of the oligonucleotide is not naturally occurring and/or the oligonucleotide comprises one or more chemical modifications or non-naturally occurring nucleotides.

An oligonucleotide of the present disclosure may comprise a number of different lengths of nucleotides. In some embodiments, the oligonucleotide is 10-100, 10-80, 10-60, 10-45, 10-40, 10-35, 10-30, 10-25, 10-20, 20-100, 20-80, 20-60, 20-45, 20-40, 20-35, 20-30, 20-25, 30-100, 30-80, 30-60, 30-45, 30-40, 30-35, 40-100, 40-80, 40-60, 40-45, 50-100, 50-80, 50-60, 60-100, 60-80, or 80-100 nucleotides in length. In some embodiments, the oligonucleotide is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 04, 95, 96, 97, 98, 99, or 100 nucleotides in length.

In some embodiments, the oligonucleotide comprises a first region that is complementary to a targeting domain of a gRNA, or portion thereof. In some embodiments, the first region of the oligonucleotide comprises at least 10 nucleotides. In some embodiments, the first region is 10-80, 10-60, 10-45, 10-40, 10-35, 10-30, 10-25, 10-20, 20-80, 20-60, 20-45, 20-40, 20-35, 20-30, 20-25, 30-80, 30-60, 30-45, 30-40, 30-35, 40-80, 40-60, 40-45, 50-80, 50-60, or 60-80 nucleotides in length. In some embodiments, the first region comprises at least 10, 11, 12, 13, 14, 15, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

In some embodiments, the first region is complementary across the full length of the targeting domain of the gRNA, e.g., for a targeting domain that is 21 nucleotides, a first region of an oligonucleotide may be complementary to the full 21 nucleotides of the targeting domain. In some embodiments, the first region is complementary across a portion of the length of the targeting domain of the gRNA. In some embodiments, the first region is complementary to at least 10%, 20%, 30%, 40%, 50%, 60%, 70%, 80%, 90%, 95%, 96%, 97%, 98%, 99% or more of the targeting domain.

In some embodiments, the oligonucleotide comprises a first region that is complementary to a targeting domain of a gRNA (i.e., fully corresponds) without mismatch. In some embodiments, the oligonucleotide comprises a first region that comprises 1 mismatch relative to the targeting domain of a gRNA. In some embodiments, the oligonucleotide comprises a first region that comprises 2 mismatches relative to the targeting domain of a gRNA. In some embodiments, the oligonucleotide comprises a first region that comprises 3 mismatches relative to the targeting domain of a gRNA. In some embodiments, the oligonucleotide comprises a first region that comprises 4 mismatches relative to the targeting domain of a gRNA. In some embodiments, the oligonucleotide comprises a first region that comprises 5 mismatches relative to the targeting domain of a gRNA.

In some embodiments, the oligonucleotide comprises a second region that is complementary to a CRISPR RNA (crRNA) sequence for a CRISPR/Cas nuclease (e.g., Cpf1 nuclease), or a portion of the crRNA. In some embodiments, the second region of the oligonucleotide comprises at least 10 nucleotides. In some embodiments, the second region is 10-80, 10-60, 10-45, 10-40, 10-35, 10-30, 10-25, 10-20, 20-80, 20-60, 20-45, 20-40, 20-35, 20-30, 20-25, 30-80, 30-60, 30-45, 30-40, 30-35, 40-80, 40-60, 40-45, 50-80, 50-60, or 60-80 nucleotides in length. In some embodiments, the first region comprises at least 10, 11, 12, 13, 14, 15, 15, 16, 17, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In some embodiments, the second region is complementary across a portion of the length of the crRNA sequence for a CRISPR/Cas nuclease (e.g., Cpf1 nuclease).

In some embodiments, the second region is complementary to at least 10%, 20%, 30%, 40%, 50%, 60%, 70%, 80%, 90%, 95%, 96%, 97%, 98%, 99% or more of the crRNA sequence for a CRISPR/Cas nuclease (e.g., Cpf1 nuclease). In some embodiments, the second region is complementary to any one or more regions of the crRNA including the proximal domain, a first complementarity domain, a linking domain, and a second complementarity domain of the crRNA.

In some embodiments, the oligonucleotide comprises a second region that is complementary to a crRNA sequence for a CRISPR/Cas nuclease (e.g., Cpf1 nuclease) (i.e., fully corresponds) without mismatch. In some embodiments, the oligonucleotide comprises a second region that comprises 1 mismatch relative to the crRNA sequence for a CRISPR/Cas nuclease (e.g., Cpf1 nuclease). In some embodiments, the oligonucleotide comprises a second region that comprises 2 mismatches relative to the crRNA sequence for a CRISPR/Cas nuclease (e.g., Cpf1 nuclease). In some embodiments, the oligonucleotide comprises a second region that comprises 3 mismatches relative to the crRNA sequence for a CRISPR/Cas nuclease (e.g., Cpf1 nuclease). In some embodiments, the oligonucleotide comprises a second region that comprises 4 mismatches relative to the crRNA sequence for a CRISPR/Cas nuclease (e.g., Cpf1 nuclease). In some embodiments, the oligonucleotide comprises a second region that comprises 5 mismatches relative to the crRNA sequence for a CRISPR/Cas nuclease (e.g., Cpf1 nuclease).

In some embodiments, the first region is 17, 18, 19, 20, 21, 22, 23, 24, 25, 26, 27, 28, 29, or 30 nucleotides in length. In some embodiments, the second region is 17, 18, 19, 20, 21, 22, 23, 24, 25, 26, 27, 28, 29, or 30 nucleotides in length.

In some embodiments, the oligonucleotide comprises 1 mismatch relative to the gRNA sequence (targeting domain and crRNA sequence). In some embodiments, the oligonucleotide comprises 2 mismatches relative to the gRNA sequence (targeting domain and crRNA sequence). In some embodiments, the oligonucleotide comprises 3 mismatches relative to the gRNA sequence (targeting domain and crRNA sequence). In some embodiments, the oligonucleotide comprises 4 mismatches relative to the gRNA sequence (targeting domain and crRNA sequence). In some embodiments, the oligonucleotide comprises 5 mismatches relative to the gRNA sequence (targeting domain and crRNA sequence).

In some embodiments, the oligonucleotide is complementary across to the gRNA sequence (targeting domain and crRNA sequence). In some embodiments, the oligonucleotide is complementary to at least 10%, 20%, 30%, 40%, 50%, 60%, 70%, 80%, 90%, 95%, 96%, 97%, 98%, 99% or more of the gRNA sequence (targeting domain and crRNA), as calculated based on the length of the oligonucleotide.

In some embodiments, the oligonucleotide does not contain more than 5 mismatches as compared to the gRNA (targeting domain and crRNA sequence). In some embodiments, the oligonucleotide is not less than 90% complementary to the gRNA (targeting domain and crRNA) as calculated based on the length of the oligonucleotide.

In some embodiments, the oligonucleotide fully binds/interacts with the targeting domain of the gRNA (binds to each of the nucleotides of the targeting domain). In some embodiments, the oligonucleotide fully binds/interacts with the crRNA sequence (binds to each of the nucleotides of the crRNA sequence). In some embodiments, the oligonucleotide fully binds/interacts with the targeting domain and the crRNA sequence (binds to each of the nucleotides of both the targeting domain and the crRNA sequence).

In some embodiments, any of the oligonucleotides provided herein comprise one or more nucleotides that are chemically modified. Chemical modifications of oligonucleotides have previously been described, and suitable chemical modifications include any modifications that are beneficial for oligonucleotide function, e.g., reduction of genomic editing at a target sequence complementary to the targeting domain of a gRNA, and do not measurably increase any undesired characteristics.

In some embodiments, the chemical modification increases the stability (e.g., the half-life) of the oligonucleotide (e.g., in a cell). In some embodiments, the chemical modification increases resistance of the oligonucleotide to a nuclease (e.g., an exonuclease or an endonuclease). In some embodiments, the chemical modification modifies a characteristic of the interaction between the oligonucleotide and a gRNA, a CRISPR/Cas nuclease (e.g., a Cpf1 nuclease), an RNP complex comprising either of the same, a target sequence (e.g., in the genome of a cell), or a combination of any thereof. For example, an oligonucleotide comprising a chemical modification may bind to a gRNA, a CRISPR/Cas nuclease (e.g., a Cpf1 nuclease), an RNP complex comprising either of the same, a target sequence (e.g., in the genome of a cell), or a combination of any thereof more strongly as compared to an oligonucleotide not comprising the chemical modification(s). In some embodiments, an oligonucleotide comprising one or more chemical modification irreversibly binds to a gRNA, a CRISPR/Cas nuclease (e.g., a Cpf1 nuclease), an RNP complex comprising either of the same, a target sequence (e.g., in the genome of a cell), or a combination of any thereof relative to the reversible binding of an oligonucleotide not comprising the chemical modification(s). In some embodiments, an oligonucleotide comprising one or more chemical modifications binds with greater specificity to a complementary sequence (e.g., to a targeting domain of a gRNA and/or a crRNA sequence) relative to an oligonucleotide not comprising the chemical modification(s).

In some embodiments, the oligonucleotide comprises one or more nucleotides comprising a chemical modification, such as a chemical modification relative to naturally occurring nucleotides. In some embodiments, at least 1, 2, 3, 4, 5, 6, 7, 8, 9, 10, 15, 20, 25, 30, 35, 40, 50, 60, 70, 80, 90, or 100 nucleotides of the oligonucleotide comprise a chemical modification. In some embodiments, at least 10, 20, 50, 75, or 100% of the nucleotides of the oligonucleotide comprise a chemical modification. In some embodiments, each nucleotide of the oligonucleotide comprises a chemical modification.

Suitable chemical modifications include, for example, those that make an oligonucleotide less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable oligonucleotide modifications will be apparent to the skilled artisan based on this disclosure and based on modification of gRNAs, such as those described for example in Rahdar et al. PNAS (2015) 112 (51) E7110-E7117 and Hendel et al., Nat Biotechnol. (2015); 33(9): 985-989, each of which is incorporated herein by reference in its entirety.

For example, an oligonucleotide provided herein may comprise one or more 2′-O modified nucleotide, e.g., a 2′-O-methyl nucleotide. In some embodiments, the oligonucleotide comprises a 2′-O modified nucleotide, e.g., 2′-O-methyl nucleotide at the 5′ end of the oligonucleotide. In some embodiments, the oligonucleotide comprises a 2′-O modified nucleotide, e.g., 2′-O-methyl nucleotide at the 3′ end of the oligonucleotide. In some embodiments, the oligonucleotide comprises a 2′-O-modified nucleotide, e.g., a 2′-O-methyl nucleotide at both the 5′ and 3′ ends of the oligonucleotide. In some embodiments, the oligonucleotide is 2′-O-modified, e.g. 2′-O-methyl-modified at the nucleotide at the 5′ end of the oligonucleotide, the second nucleotide from the 5′ end of the oligonucleotide, and the third nucleotide from the 5′ end of the oligonucleotide. In some embodiments, the oligonucleotide is 2′-O-modified, e.g., 2′-O-methyl-modified at the nucleotide at the 3′ end of the oligonucleotide, the second nucleotide from the 3′ end of the oligonucleotide, and the third nucleotide from the 3′ end of the oligonucleotide. In some embodiments, the oligonucleotide is 2′-O-modified, e.g., 2′-O-methyl-modified at the nucleotide at the 5′ end of the oligonucleotide, the second nucleotide from the 5′ end of the oligonucleotide, the third nucleotide from the 5′ end of the oligonucleotide, the nucleotide at the 3′ end of the oligonucleotide, the second nucleotide from the 3′ end of the oligonucleotide, and the third nucleotide from the 3′ end of the oligonucleotide. In some embodiments, the oligonucleotide is 2′-O-modified, e.g., 2′-O-methyl-modified at the second nucleotide from the 3′ end of the oligonucleotide, the third nucleotide from the 3′ end of the oligonucleotide, and at the fourth nucleotide from the 3′ end of the oligonucleotide. In some embodiments, the nucleotide at the 3′ end of the oligonucleotide is not chemically modified. In some embodiments, the nucleotide at the 3′ end of the oligonucleotide does not have a chemically modified sugar. In some embodiments, the oligonucleotide is 2′-O-modified, e.g., 2′-O-methyl-modified, at the nucleotide at the 5′ end of the oligonucleotide, the second nucleotide from the 5′ end of the oligonucleotide, the third nucleotide from the 5′ end of the oligonucleotide, the second nucleotide from the 3′ end of the oligonucleotide, the third nucleotide from the 3′ end of the oligonucleotide, and the fourth nucleotide from the 3′ end of the oligonucleotide. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, an oligonucleotide provided herein may comprise one or more 2′-O-modified and 3′phosphorous-modified nucleotide, e.g., a 2′-O-methyl 3′phosphorothioate nucleotide. In some embodiments, the oligonucleotide comprises a 2′-O-modified and 3′phosphorous-modified, e.g., 2′-O-methyl 3′phosphorothioate nucleotide at the 5′ end of the oligonucleotide. In some embodiments, the oligonucleotide comprises a 2′-O-modified and 3′phosphorous-modified, e.g., 2′-O-methyl 3′phosphorothioate nucleotide at the 3′ end of the oligonucleotide. In some embodiments, the oligonucleotide comprises a 2′-O-modified and 3′phosphorous-modified, e.g., 2′-O-methyl 3′phosphorothioate nucleotide at the 5′ and 3′ ends of the gRNA. In some embodiments, the oligonucleotide comprises a backbone in which one or more non-bridging oxygen atoms has been replaced with a sulfur atom. In some embodiments, the oligonucleotide is 2′-O-modified and 3′phosphorous-modified, e.g. 2′-O-methyl 3′phosphorothioate-modified at the nucleotide at the 5′ end of the oligonucleotide, the second nucleotide from the 5′ end of the oligonucleotide, and the third nucleotide from the 5′ end of the oligonucleotide. In some embodiments, the oligonucleotide is 2′-O-modified and 3′phosphorous-modified, e.g. 2′-O-methyl 3′phosphorothioate-modified at the nucleotide at the 3′ end of the oligonucleotide, the second nucleotide from the 3′ end of the oligonucleotide, and the third nucleotide from the 3′ end of the oligonucleotide. In some embodiments, the oligonucleotide is 2′-O-modified and 3′phosphorous-modified, e.g. 2′-O-methyl 3′phosphorothioate-modified at the nucleotide at the 5′ end of the oligonucleotide, the second nucleotide from the 5′ end of the oligonucleotide, the third nucleotide from the 5′ end of the oligonucleotide, the nucleotide at the 3′ end of the gRNA, the second nucleotide from the 3′ end of the oligonucleotide, and the third nucleotide from the 3′ end of the oligonucleotide. In some embodiments, the oligonucleotide is 2′-O-modified and 3′phosphorous-modified, e.g. 2′-O-methyl 3′phosphorothioate-modified at the second nucleotide from the 3′ end of the oligonucleotide, the third nucleotide from the 3′ end of the oligonucleotide, and the fourth nucleotide from the 3′ end of the oligonucleotide. In some embodiments, the nucleotide at the 3′ end of the oligonucleotide is not chemically modified. In some embodiments, the nucleotide at the 3′ end of the oligonucleotide does not have a chemically modified sugar. In some embodiments, the oligonucleotide is 2′-O-modified and 3′phosphorous-modified, e.g. 2′-O-methyl 3′phosphorothioate-modified at the nucleotide at the 5′ end of the gRNA, the second nucleotide from the 5′ end of the oligonucleotide, the third nucleotide from the 5′ end of the oligonucleotide, the second nucleotide from the 3′ end of the oligonucleotide, the third nucleotide from the 3′ end of the oligonucleotide, and the fourth nucleotide from the 3′ end of the oligonucleotide.

In some embodiments, an oligonucleotide provided herein may comprise one or more 2′-O-modified and 3′-phosphorous-modified, e.g., 2′-O-methyl 3′thioPACE nucleotide. In some embodiments, the oligonucleotide comprises a 2′-O-modified and 3′phosphorous-modified, e.g., 2′-O-methyl 3′thioPACE nucleotide at the 5′ end of the oligonucleotide. In some embodiments, the oligonucleotide comprises a 2′-O-modified and 3′phosphorous-modified, e.g., 2′-O-methyl 3′thioPACE nucleotide at the 3′ end of the oligonucleotide. In some embodiments, the oligonucleotide comprises a 2′-O-modified and 3′phosphorous-modified, e.g., 2′-O-methyl 3′thioPACE nucleotide at the 5′ and 3′ ends of the oligonucleotide. In some embodiments, the oligonucleotide comprises a backbone in which one or more non-bridging oxygen atoms have been replaced with a sulfur atom and one or more non-bridging oxygen atoms have been replaced with an acetate group. In some embodiments, the oligonucleotide is 2′-O-modified and 3′phosphorous-modified, e.g. 2′-O-methyl 3′ thioPACE-modified at the nucleotide at the 5′ end of the oligonucleotide, the second nucleotide from the 5′ end of the oligonucleotide, and the third nucleotide from the 5′ end of the oligonucleotide. In some embodiments, the oligonucleotide is 2′-O-modified and 3′phosphorous-modified, e.g. 2′-O-methyl 3′thioPACE-modified at the nucleotide at the 3′ end of the oligonucleotide, the second nucleotide from the 3′ end of the oligonucleotide, and the third nucleotide from the 3′ end of the oligonucleotide. In some embodiments, the oligonucleotide is 2′-O-modified and 3′phosphorous-modified, e.g. 2′-O-methyl 3′thioPACE-modified at the nucleotide at the 5′ end of the gRNA, the second nucleotide from the 5′ end of the oligonucleotide, the third nucleotide from the 5′ end of the oligonucleotide, the nucleotide at the 3′ end of the oligonucleotide, the second nucleotide from the 3′ end of the oligonucleotide, and the third nucleotide from the 3′ end of the oligonucleotide. In some embodiments, the oligonucleotide is 2′-O-modified and 3′phosphorous-modified, e.g. 2′-O-methyl 3′thioPACE-modified at the second nucleotide from the 3′ end of the oligonucleotide, the third nucleotide from the 3′ end of the oligonucleotide, and the fourth nucleotide from the 3′ end of the oligonucleotide. In some embodiments, the nucleotide at the 3′ end of the oligonucleotide is not chemically modified. In some embodiments, the nucleotide at the 3′ end of the oligonucleotide does not have a chemically modified sugar. In some embodiments, the oligonucleotide is 2′-O-modified and 3′phosphorous-modified, e.g. 2′-O-methyl 3′thioPACE-modified at the nucleotide at the 5′ end of the oligonucleotide, the second nucleotide from the 5′ end of the oligonucleotide, the third nucleotide from the 5′ end of the oligonucleotide, the second nucleotide from the 3′ end of the oligonucleotide, the third nucleotide from the 3′ end of the oligonucleotide, and the fourth nucleotide from the 3′ end of the oligonucleotide.

In some embodiments, an oligonucleotide provided herein comprises a chemically modified backbone. In some embodiments, the oligonucleotide comprises a phosphorothioate linkage. In some embodiments, one or more non-bridging oxygen atoms have been replaced with a sulfur atom. In some embodiments, the nucleotide at the 5′ end of the oligonucleotide, the second nucleotide from the 5′ end of the oligonucleotide, and the third nucleotide from the 5′ end of the oligonucleotide each comprise a phosphorothioate linkage. In some embodiments, the nucleotide at the 3′ end of the oligonucleotide, the second nucleotide from the 3′ end of the oligonucleotide, and the third nucleotide from the 3′ end of the oligonucleotide each comprise a phosphorothioate linkage. In some embodiments, the nucleotide at the 5′ end of the oligonucleotide, the second nucleotide from the 5′ end of the oligonucleotide, the third nucleotide from the 5′ end of the oligonucleotide, the nucleotide at the 3′ end of the oligonucleotide, the second nucleotide from the 3′ end of the oligonucleotide, and the third nucleotide from the 3′ end of the oligonucleotide each comprise a phosphorothioate linkage. In some embodiments, the second nucleotide from the 3′ end of the oligonucleotide, the third nucleotide from the 3′ end of the oligonucleotide, and at the fourth nucleotide from the 3′ end of the oligonucleotide each comprise a phosphorothioate linkage. In some embodiments, the nucleotide at the 5′ end of the oligonucleotide, the second nucleotide from the 5′ end of the oligonucleotide, the third nucleotide from the 5′ end of the oligonucleotide, the second nucleotide from the 3′ end of the oligonucleotide, the third nucleotide from the 3′ end of the oligonucleotide, and the fourth nucleotide from the 3′ end of the oligonucleotide each comprise a phosphorothioate linkage.

In some embodiments, the oligonucleotide comprises a phosphorothioate (i.e., a phosphorothioate linkage or a phosphorothioate backbone linkage). In some embodiments, at least 10, 20, 30, 40, 50, 60, 70, 80, 90, or 100% of the nucleotides of the oligonucleotide comprise a phosphorothioate linkage. In some embodiments, at least 1, 2, 5, 10, 15, 20, 25, 30, 35, or 40 nucleotides of the oligonucleotide comprise a phosphorothioate linkage. In some embodiments, each nucleotide of an oligonucleotide comprises a phosphorothioate linkage.

In some embodiments, an oligonucleotide provided herein comprises a thioPACE linkage. In some embodiments, the oligonucleotide comprises a backbone in which one or more non-bridging oxygen atoms have been replaced with a sulfur atom and one or more non-bridging oxygen atoms have been replaced with an acetate group. In some embodiments, the nucleotide at the 5′ end of the oligonucleotide, the second nucleotide from the 5′ end of the oligonucleotide, and the third nucleotide from the 5′ end of the oligonucleotide each comprise a thioPACE linkage. In some embodiments, the nucleotide at the 3′ end of the oligonucleotide, the second nucleotide from the 3′ end of the oligonucleotide, and the third nucleotide from the 3′ end of the oligonucleotide each comprise a thioPACE linkage. In some embodiments, the nucleotide at the 5′ end of the oligonucleotide, the second nucleotide from the 5′ end of the oligonucleotide, the third nucleotide from the 5′ end of the oligonucleotide, the nucleotide at the 3′ end of the oligonucleotide, the second nucleotide from the 3′ end of the oligonucleotide, and the third nucleotide from the 3′ end of the oligonucleotide each comprise a thioPACE linkage. In some embodiments, the second nucleotide from the 3′ end of the oligonucleotide, the third nucleotide from the 3′ end of the oligonucleotide, and at the fourth nucleotide from the 3′ end of the oligonucleotide each comprise a thioPACE linkage. In some embodiments, the nucleotide at the 5′ end of the oligonucleotide, the second nucleotide from the 5′ end of the oligonucleotide, the third nucleotide from the 5′ end of the oligonucleotide, the second nucleotide from the 3′ end of the oligonucleotide, the third nucleotide from the 3′ end of the oligonucleotide, and the fourth nucleotide from the 3′ end of the oligonucleotide each comprise a thioPACE linkage.

In some embodiments, an oligonucleotide described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, an oligonucleotide described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the nucleotide at the 5′ end of the oligonucleotide, the second nucleotide from the 5′ end of the oligonucleotide, the third nucleotide from the 5′ end of the oligonucleotide, the second nucleotide from the 3′ end of the oligonucleotide, the third nucleotide from the 3′ end of the oligonucleotide, and the fourth nucleotide from the 3′ end of the oligonucleotide each comprise a 2′-O-methyl-3′-phosphorothioate nucleotides. In some embodiments, the oligonucleotide may comprise one or more modified nucleotides, e.g., as described in PCT Publication Nos. WO 2017/214460, WO 2016/089433, and WO 2016/164356, which are incorporated by reference their entirety.

In some embodiments, an oligonucleotide described herein comprises one or more locked nucleic acids (LNA), e.g., at least 1, 2, 3, 4, 5, or 6 locked nucleic acids. In some embodiments, the oligonucleotide comprises a locked nucleic acid. In some embodiments, at least 10, 20, 30, 40, 50, 60, 70, 80, 90, or 100% of the nucleotides of the oligonucleotide is a locked nucleic acid. In some embodiments, at least 1, 2, 5, 10, 15, 20, 25, 30, 35, or 40 nucleotides of the oligonucleotide is a locked nucleic acid. In some embodiments, each nucleotide of an oligonucleotide is a locked nucleic acid.

As described herein, the oligonucleotides are capable of reducing genomic editing at a target sequence complementary to the targeting domain of a gRNA, where the target sequence and targeting domain of the gRNA may be any corresponding nucleic acid sequences (e.g., targeting any sequence in the genome of a cell). In some embodiments, an oligonucleotide comprises a first region that is complementary to targeting domain of a gRNA provided in SEQ ID NOs: 3-6.

Exemplary oligonucleotides are provided herein. In some embodiments, an oligonucleotide comprises a nucleic acid sequence of any of SEQ ID NOs: 1-2, or a sequence having no more than 1, 2, 3, 4, 5, 6, 7, 8, 9, or 10 alterations relative thereto.

In some embodiments, an oligonucleotide comprises a second region that is complementary to a CRISPR RNA (crRNA) sequence for a CRISPR/Cas nuclease (e.g., a Cpf1 nuclease). wherein the second region comprises a nucleic acid sequence of SEQ ID NO: 13, or a sequence having no more than 1, 2, 3, 4, 5, 6, 7, 8, 9, or 10 alterations relative thereto.

An exemplary nucleotide sequence of a region that is complementary to a crRNA sequence is provided by SEQ ID NO: 13 below.

(SEQ ID NO: 13)
5′-ATCTACAAGAGTAGAAATTA-3′

Exemplary oligonucleotides of the disclosure are provided below. Lowercase letters indicate the first region of the oligonucleotide that is complementary to the targeting domain of an exemplary gRNA with a targeting sequence in the listed exemplary target gene. Uppercase letters indicate the sequence of the second region of the oligonucleotide that is complementary to an exemplary crRNA sequence compatible with a Cpf1 nuclease, e.g., AsCpf1.

TABLE A
Exemplary oligonucleotide sequences

| Target | Nuclease | Sequence (5′-3′) |
|---|---|---|
| CCR5 | AsCpf1 | t*g*c*t*g*g*t*c*a*t*c*c*t*c*a*t*c*c*t*g*a*A*T |
| (cc18) | | *C*T*A*C*A*A*G*A*G*T*A*G*A*A*A*T*T*A |
| | | (SEQ ID NO: 1) |
| CD19 | AsCpf1 | c*t*g*g*t*c*t*c*g*g*g*a*g*t*c*c*c*c*g*c*t*A*T |
| (g3) | | *C*T*A*C*A*A*G*A*G*T*A*G*A*A*A*T*T*A |
| | | (SEQ ID NO: 2) |

In Table A, “*” refers to the location of a phosphorothioate linkage.

### CRISPR/Cas Systems

Some aspects of this disclosure provide compositions and methods for generating the genetically engineered cells described herein. One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, e.g., Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an spCas9 or an saCas9 nuclease. For another example, in some embodiments, a suitable RNA-guided nuclease for use in the methods of genetically engineering cells provided herein is a Cas12 nuclease, e.g., a Cas12a nuclease (also referred to as a “Cpf1 nuclease”). As used herein, a Cpf1 nuclease refers to a polypeptide i) derived from a type II class 2 CRISPR/Cas nuclease that cleaves distal to a PAM site, and ii) capable of, in combination with a suitable gRNA, binding a target nucleic acid sequence (a target sequence). In some embodiments, a Cpf1 nuclease is capable of producing a single-stranded break in DNA. In some embodiments, a Cpf1 nuclease is capable of producing a double-stranded break in DNA. In some embodiments, a Cpf1 nuclease lacks or has reduced nuclease activity. In some embodiments, a Cpf1 nuclease comprises an amino acid sequence of SEQ ID NO: 12, or comprises an amino acid sequence with at least 80, 85, 90, 91, 92, 93, 94, 95, 96, 97, 98, or 99% identity to any thereof.

Exemplary suitable Cas12 nucleases include, without limitation, AsCas12a, FnCas12a, LbCas12a, PaCas12a. other Cas12a orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816; PCT Publication Nos. WO 2016/166340; WO 2017/155407; WO 2018/083128; WO 2016/205711; WO 2017/035388; WO 2017/184768; WO2019/118516; WO2017/184768; WO 2018/098383; WO 2020/146297; and WO 2020/172502. In some embodiments, a gRNA described herein is suitable for use with a Cpf1 nuclease.

The amino acid sequence of an exemplary Cpf1 nuclease (from Acidaminococcus sp. is provided by SEQ ID NO: 12 below (UniProt U2UMQ6).

MTQFEGFTNLYQVSKTLRFELIPQGKTLKHIQEQGFIEEDKARNDHYKE
LKPIIDRIYKTYADQCLQLVQLDWENLSAAIDSYRKEKTEETRNALIEE
QATYRNAIHDYFIGRTDNLTDAINKRHAEIYKGLFKAELFNGKVLKQLG
TVTTTEHENALLRSFDKFTTYFSGFYENRKNVFSAEDISTAIPHRIVQD
NFPKFKENCHIFTRLITAVPSLREHFENVKKAIGIFVSTSIEEVFSFPF
YNQLLTQTQIDLYNQLLGGISREAGTEKIKGLNEVLNLAIQKNDETAHI
IASLPHRFIPLFKQILSDRNTLSFILEEFKSDEEVIQSFCKYKILLRNE
NVLETAEALFNELNSIDLTHIFISHKKLETISSALCDHWDTLRNALYER
RISELTGKITKSAKEKVQRSLKHEDINLQEIISAAGKELSEAFKQKTSE
ILSHAHAALDQPLPTTLKKQEEKEILKSQLDSLLGLYHLLDWFAVDESN
EVDPEFSARLTGIKLEMEPSLSFYNKARNYATKKPYSVEKFKLNFQMPT
LASGWDVNKEKNNGAILFVKNGLYYLGIMPKQKGRYKALSFEPTEKTSE
GFDKMYYDYFPDAAKMIPKCSTQLKAVTAHFQTHTTPILLSNNFIEPLE
ITKEIYDLNNPEKEPKKFQTAYAKKTGDQKGYREALCKWIDFTRDFLSK
YTKTTSIDLSSLRPSSQYKDLGEYYAELNPLLYHISFQRIAEKEIMDAV
ETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHR
LSHDLSDEARALLPNVITKEVSHEIIKDRRFTSDKFFFHVPITLNYQAA
NSPSKFNQRVNAYLKEHPETPIIGIDRGERNLIYITVIDSTGKILEQRS
LNTIQQFDYQKKLDNREKERVAARQAWSVVGTIKDLKQGYLSQVIHEIV
DLMIHYQAVVVLENLNFGFKSKRTGIAEKAVYQQFEKMLIDKLNCLVLK
DYPAEKVGGVLNPYQLTDQFTSFAKMGTQSGFLFYVPAPYTSKIDPLTG
FVDPFVWKTIKNHESRKHFLEGFDFLHYDVKTGDFILHFKMNRNLSFQR
GLPGFMPAWDIVFEKNETQFDAKGTPFIAGKRIVPVIENHRFTGRYRDL
YPANELIALLEEKGIVFRDGSNILPKLLENDDSHAIDTMVALIRSVLQM
RNSNAATGEDYINSPVRDLNGVCFDSRFQNPEWPMDADANGAYHIALKG
QLLLNHLKESKDLKLQNGISNQDWLAYIQELRN

In some embodiments, a genetically engineered cell described herein is generated by targeting an RNA-guided nuclease, e.g., a CRISPR/Cas nuclease, such as, for example, a Cas12a nuclease (e.g., Cpf1) or a Cas9 nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs (i.e., gRNAs) are described in more detail elsewhere herein.

In some embodiments, any of the gRNAs described herein may be complexed with a suitable CRISPR/Cas nuclease. Exemplary suitable nucleases include, for example, Cas12a (Cpf1) nucleases and Cas 9 nucleases, including e.g., base editing nucleases.

Various Cas9 nucleases are suitable for use with the gRNAs provided herein to effect genome editing according to aspects of this disclosure, e.g., to create a genomic modification in a target genetic loci. Typically, the CRISPR/Cas nuclease and the gRNA are provided in a form and under conditions suitable for the formation of a nuclease/gRNA complex (i.e., a CRISPR system), which may be referred to as a ribonucleoprotein (RNP) complex, that targets a target site on the genome of the cell. In some embodiments, a CRISPR/Cas nuclease is used that exhibits a desired PAM specificity to target the nuclease/gRNA complex to a desired target site sequence in a genetic loci. Example target domains and corresponding gRNA targeting domain sequences are provided herein.

In some embodiments, a nuclease/gRNA complex is formed, e.g., in vitro, and a target cell is contacted with the nuclease/gRNA complex, e.g., via electroporation of the nuclease/gRNA complex into the cell. In some embodiments, the cell is contacted with the CRISPR/Cas protein and gRNA separately, and the nuclease/gRNA complex is formed within the cell. In some embodiments, the cell is contacted with a nucleic acid, e.g., a DNA or RNA, encoding the CRISPR/Cas protein, and/or with a nucleic acid encoding the gRNA, or both.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71: 1-9.

In some embodiments, genetically engineered cells as provided herein are generated using a suitable genome editing technology, wherein the genome editing technology is characterized by the use of a Cas12a (Cpf1) nuclease.

In some embodiments, a CRISPR/Cas nuclease used in the methods of genome editing provided herein is a Cas12a (Cpf1) nuclease derived from Provetella spp. (e.g., Provetella ihumii (PiCas12a/PiCpf1) or Provetella disiens (PdCas12a/PdCpf1) or Francisella spp. (Francisella novicida (FnCas12a/FnCpf1)), Acidaminococcus sp. (AsCas12a/AsCpf1), Lachnospiraceae bacterium (LbCas12a/LbCpf1), or Eubacterium rectale (ErCas12a/ErCpf1). In some embodiments, the CRISPR/Cas nuclease is MAD7™ (Inscripta, Inc.). In some embodiments, the CRISPR/Cas nuclease is MAD7™ from Eubacterium rectale (ErMAD7).

Use of Cas12a and MAD7 enzymes for genome editing are well known in the art. See, e.g., Wierson et al. CRISPR J. (2019) 6: 417-433; Price et al. Biotechnol. Bioeng. (2020) 6: 1805-1816.

The amino acid sequence of an exemplary MAD7 nuclease from Eubacterium rectale (GenScript™) is provided by SEQ ID NO: 14 below:

(SEQ ID NO: 14)
MNNGTNNFQNFIGISSLQKTLRNALIPTETTQQFIVKNGIIKEDELRGE
NRQILKDIMDDYYRGFISETLSSIDDIDWTSLFEKMEIQLKNGDNKDTL
IKEQTEYRKAIHKKFANDDRFKNMFSAKLISDILPEFVIHNNNYSASEK
EEKTQVIKLESRFATSFKDYFKNRANCFSADDISSSSCHRIVNDNAEIF
FSNALVYRRIVKSLSNDDINKISGDMKDSLKEMSLEEIYSYEKYGEFIT
QEGISFYNDICGKVNSFMNLYCQKNKENKNLYKLQKLHKQILCIADTSY
EVPYKFESDEEVYQSVNGFLDNISSKHIVERLRKIGDNYNGYNLDKIYI
VSKFYESVSQKTYRDWETINTALEIHYNNILPGNGKSKADKVKKAVKND
LQKSITEINELVSNYKLCSDDNIKAETYIHEISHILNNFEAQELKYNPE
IHLVESELKASELKNVLDVIMNAFHWCSVFMTEELVDKDNNFYAELEEI
YDEIYPVISLYNLVRNYVTQKPYSTKKIKLNFGIPTLADGWSKSKEYSN
NAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLI
DYFKNCIAIHPEWKNFGFDFSDTSTYEDISGFYREVELQGYKIDWTYIS
EKDIDLLQEKGQLYLFQIYNKDFSKKSTGNDNLHTMYLKNLFSEENLKD
IVLKLNGEAEIFFRKSSIKNPIIHKKGSILVNRTYEAEEKDQFGNIQIV
RKNIPENIYQELYKYFNDKSDKELSDEAAKLKNVVGHHEAATNIVKDYR
YTYDKYFLHMPITINFKANKTGFINDRILQYIAKEKDLHVIGIDRGERN
LIYVSVIDTCGNIVEQKSFNIVNGYDYQIKLKQQEGARQIARKEWKEIG
KIKEIKEGYLSLVIHEISKMVIKYNAIIAMEDLSYGFKKGRFKVERQVY
QKFETMLINKLNYLVFKDISITENGGLLKGYQLTYIPDKLKNVGHQCGC
IFYVPAAYTSKIDPTTGFVNIFKFKDLTVDAKREFIKKFDSIRYDSEKN
LFCFTFDYNNFITQNTVMSKSSWSVYTYGVRIKRRFVNGRFSNESDTID
ITKDMEKTLEMTDINWRDGHDLRQDIIDYEIVQHIFEIFRLTVQMRNSL
SELEDRDYDRLISPVLNENNIFYDSAKAGDALPKDADANGAYCIALKGL
YEIKQITENWKEDGKFSRDKLKISNKDWFDFIQNKRYLKRPAATKKAGQ
AKKKKGSYPYDVPDYAYPYDVPDYAYPYDVPDYAGSPKKKRKVGSHHHH
HH

Alternatively, the amino acid of an exemplary MAD7 nuclease (Inscripta™) is provided by SEQ ID NO: 15 below:

(SEQ ID NO: 15)
MNNGTNNFQNFIGISSLQKTLRNALIPTETTQQFIVKNGIIKEDELRGE
NRQILKDIMDDYYRGFISETLSSIDDIDWTSLFEKMEIQLKNGDNKDTL
IKEQTEYRKAIHKKFANDDRFKNMFSAKLISDILPEFVIHNNNYSASEK
EEKTQVIKLFSRFATSFKDYFKNRANCFSADDISSSSCHRIVNDNAEIF
FSNALVYRRIVKSLSNDDINKISGDMKDSLKEMSLEEIYSYEKYGEFIT
QEGISFYNDICGKVNSFMNLYCQKNKENKNLYKLQKLHKQILCIADTSY
EVPYKFESDEEVYQSVNGFLDNISSKHIVERLRKIGDNYNGYNLDKIYI
VSKFYESVSQKTYRDWETINTALEIHYNNILPGNGKSKADKVKKAVKND
LQKSITEINELVSNYKLCSDDNIKAETYIHEISHILNNFEAQELKYNPE
IHLVESELKASELKNVLDVIMNAFHWCSVFMTEELVDKDNNFYAELEEI
YDEIYPVISLYNLVRNYVTQKPYSTKKIKLNFGIPTLADGWSKSKEYSN
NAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLI
DYFKNCIAIHPEWKNFGFDFSDTSTYEDISGFYREVELQGYKIDWTYIS
EKDIDLLQEKGQLYLFQIYNKDFSKKSTGNDNLHTMYLKNLFSEENLKD
IVLKLNGEAEIFFRKSSIKNPIIHKKGSILVNRTYEAEEKDQFGNIQIV
RKNIPENIYQELYKYFNDKSDKELSDEAAKLKNVVGHHEAATNIVKDYR
YTYDKYFLHMPITINFKANKTGFINDRILQYIAKEKDLHVIGIDRGERN
LIYVSVIDTCGNIVEQKSFNIVNGYDYQIKLKQQEGARQIARKEWKEIG
KIKEIKEGYLSLVIHEISKMVIKYNAIIAMEDLSYGFKKGRFKVERQVY
QKFETMLINKLNYLVFKDISITENGGLLKGYQLTYIPDKLKNVGHQCGC
IFYVPAAYTSKIDPTTGFVNIFKFKDLTVDAKREFIKKFDSIRYDSEKN
LFCFTFDYNNFITQNTVMSKSSWSVYTYGVRIKRRFVNGRFSNESDTID
ITKDMEKTLEMTDINWRDGHDLRQDIIDYEIVQHIFEIFRLTVQMRNSL
SELEDRDYDRLISPVLNENNIFYDSAKAGDALPKDADANGAYCIALKGL
YEIKQITENWKEDGKFSRDKLKISNKDWEDFIQNKRYL

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (StCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

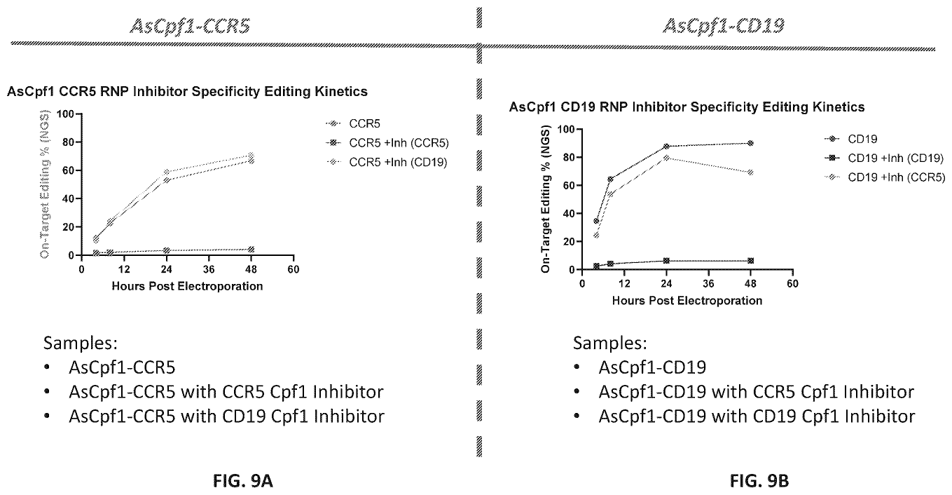

A naturally occurring Cas9 nuclease typically comprises two lobes: a recognition (REC) lobe and a nuclease (NUC) lobe; each of which further comprises domains described, e.g., in PCT Publication No. WO 2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science (2014) 343(6176): 1247997) and for S. pyogenes Cas9 with a guide RNA (e.g., a synthetic fusion of crRNA and tracrRNA) (e.g., Nishimasu et al., Cell (2014) 156:935-949; and Anders et al., Nature (2014), doi: 10.1038/naturel3579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various CRISPR/Cas nucleases, which may also be referred to as Cas nucleases, are known in the art and may be obtained from various sources and/or engineered/modified to modulate one or more activities or specificities of the enzymes. PAM sequence preferences and specificities of suitable Cas nucleases are known in the art. In some embodiments, the PAM sequence preferences and specificities of suitable Cas nucleases, e.g., suitable Cas9 nucleases, such as, for example, spCas9 and saCas9, are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a CRISPR/Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35(8): 789-792.

In some embodiments, a base editor is used to create a genomic modification in a cell. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive CRISPR/Cas nuclease is referred to as “dead Cas”, “dCas”, or “dead CRISPR/Cas nuclease.” In some embodiments, the endonuclease comprises a dCas fused to an adenine base editor (ABE), for example an ABE evolved from the RNA adenine deaminase TadA. In some embodiments, the endonuclease comprises a dCas fused to cytidine deaminase enzyme (e.g., APOBEC deaminase, pmCDA1, activation-induced cytidine deaminase (AID)). In some embodiments, the catalytically inactive Cas molecule has reduced activity and is, e.g., a nickase.

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in U.S. Publication No. 2018/0312825A1, U.S. Publication No. 2018/0312828A1, and PCT Publication No. WO 2018/165629A1, which are incorporated by reference herein in their entireties.

Some aspects of this disclosure provide guide RNAs that are suitable to target an RNA-guided nuclease, e.g. as provided herein, to a suitable target site in the genome of a cell. In some embodiments, the gRNA effects a modification in the genome of the cell (e.g., insertion, mutation, deletion). Such modifications may result in a loss of expression and/or regulation of a protein encoded by a gene, or expression of a variant form of a protein encoded by a genet that is targeted by the gRNA.

The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNA sequence).

Suitable gRNAs for use with CRISPR/Cas nucleases, such as Cas12a nucleases, typically comprise a single RNA molecule, as the naturally occurring Cas12a guide RNA comprises a single RNA molecule. A suitable gRNA may thus be unimolecular (having a single RNA molecule), sometimes referred to herein as single guide RNAs (sgRNAs), or modular (comprising more than one, and typically two, separate RNA molecules). Some exemplary suitable Cas12a gRNA scaffold sequences are provided herein, and additional suitable gRNA scaffold sequences will be apparent to the skilled artisan based on the present disclosure.

In some embodiments, e.g., in some embodiments where a Cas12a nuclease is used, a gRNA, may comprise, from 5′ to 3′:
a CRISPR RNA (crRNA) sequence for a CRISPR/Cas nuclease, containing:
   a proximal domain;
   a first complementarity domain;
   a linking domain; and
   a second complementarity domain (which is complementary to the first complementarity domain); and
a targeting domain corresponding to a target site sequence.

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO 2014/093694, and PCT Publication No. WO 2013/176772, incorporated herein by reference.

For example, the binding domains of naturally occurring spCas9 gRNA typically comprise two RNA molecules, the crRNA (partially) and the tracrRNA. Variants of spCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” A gRNA suitable for targeting a target site may comprise a number of domains. In some embodiments, e.g., in some embodiments where a Cas9 nuclease is used, a unimolecular sgRNA, may comprise, from 5′ to 3′:
a targeting domain corresponding to a target site sequence in a target locus;
a first complementarity domain;
a linking domain;
a second complementarity domain (which is complementary to the first complementarity domain);
a proximal domain; and
optionally, a tail domain.

Each of these domains is now described in more detail.

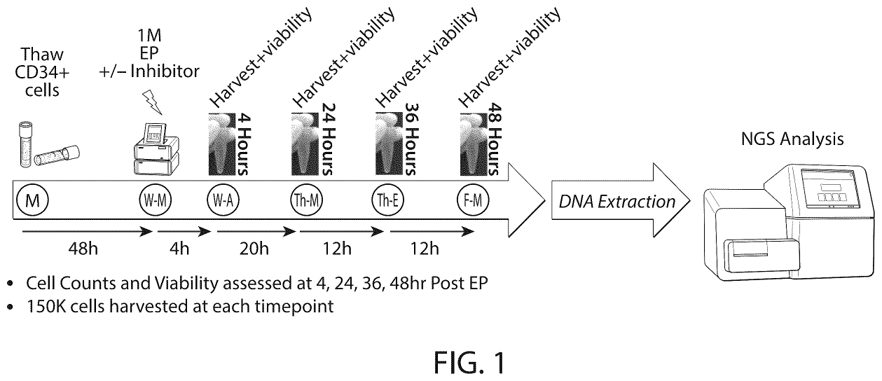

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., Figure 1 of Vanegas et al., Fungal Biol Biotechnol. (2019) 6: 6, which is incorporated by reference herein. For additional illustration and description of the mechanism of gRNA targeting an RNA-guided nuclease to a target site, see Fu Y et al, Nat Biotechnol (2014) (doi: 10.1038/nbt.2808) and Sternberg S H et al., Nature (2014) (doi: 10.1038/naturel3011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

The structure of a typical Cas12a gRNA can be found, for example in Figure 1 of Zetsche et al. Cell (2015) 163(3): 759-771, which is incorporated by reference herein in its entirety. An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
                          [PAM] [ target domain (DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
      [binding domain][ targeting domain (RNA) ]
```

In some embodiments, the Cas12a PAM sequence is 5′-T-T-T-V-3′. In some embodiments, the Cas12a PAM sequence is 5′-T-T-V-3′.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

```
      [ target domain (DNA) ] [ PAM ]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
      | | | | | | | | | | | | | | | | | | | | | |
5′-N-N-N-N-N-N-N-N-N-N-N-N-N-N-N-N-N-N-N-N-N-N-[gRNA scaffold]-3′ (RNA)
      [ targeting domain (RNA) ] [binding domain]
```

While not wishing to be bound by theory, at least in some embodiments, it is believed that the length and complementarity of the targeting domain with the target sequence contributes to specificity of the interaction of the gRNA/Cas molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO 2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO 2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO 2018/126176, the entire contents of which are incorporated herein by reference.

In some embodiments, the second complementarity domain of the targeting domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
   a targeting domain (which corresponds to a target domain in a target locus); and
   a first complementarity domain; and
a second strand, comprising, e.g., from 5′ to 3′:
   optionally, a 5′ extension domain;
   a second complementarity domain;
   a proximal domain; and
   optionally, a tail domain.

Exemplary targeting domains for gRNAs suitable for use in the methods and compositions of the disclosure are provided below.

TABLE B
Exemplary targeting domain sequences

| Target | Nuclease | Sequence (5′-3′) |
|---|---|---|
| CCR5 (cc18) | AsCpf1 | TCAGGATGAGGATGACCAGCA (SEQ ID NO: 3) |
| CD19 (g3) | AsCpf1 | AGCGGGGACTCCCGAGACCAG (SEQ ID NO: 4) |
| CD5 (g137) | SpCas9 | CATAGCTGATGGTACCCCCC (SEQ ID NO: 5) |
| CD33 (g60) | SpCas9 | GGTGGGGGCAGCTGACAACC (SEQ ID NO: 6) |

In Table B, “AsCpf1” refers to Cpf1 nuclease from Acidominococcus sp., and “SpCas9” refers to Cas9 nuclease from Streptococcus pyogenes.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g., 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g., 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a 2′-O-methyl-3′-phosphorothioate nucleotides. In some embodiments, the gRNA may comprise one or more modified nucleotides, e.g., as described in PCT Publication Nos. WO 2017/214460, WO 2016/089433, and WO 2016/164356, which are incorporated by reference their entirety.

The gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

In some embodiments, the gRNAs described herein are capable of directing a CRISPR/Cas nuclease to a target site sequence and directing cleavage of one or both strands of DNA at the target site sequence.

Genetically engineering cells and compositions comprising or associated with said cells Aspects of the present disclosure provide methods for effecting a genetic modification (e.g., mutation) in the genome of a cell, in a controllable/regulatable manner using oligonucleotides that prevent, reduce, or eliminate CRISPR/Cas nuclease (e.g., Cpf1 nuclease) activity, e.g., in a gRNA or target sequence specific manner. The oligonucleotides described herein comprise a first region that is complementary to a targeting domain of a gRNA and a second region that is complementary to a CRISPR RNA (crRNA) sequence for a CRISPR/Cas nuclease (e.g., Cpf1 nuclease). Without wishing to be bound by theory, the oligonucleotide may bind to one or more of a gRNA, an RNP complex comprising a gRNA and a CRISPR/Cas nuclease (e.g., Cpf1 nuclease), a gRNA and its target site sequence (e.g., in the genome of the cell), or an RNP complex comprising a gRNA, CRISPR/Cas nuclease (e.g., Cpf1 nuclease), and the target sequence of the gRNA. Consequently, the oligonucleotide may reduce genomic editing by a CRISPR/Cas nuclease (e.g., Cpf1 nuclease) guided by the gRNA and/or at the target sequence of the gRNA (e.g., by reducing or eliminating interaction between members of the RNP complex or the RNP complex and target site sequence, inhibiting formation or maintenance of the RNP complex, and/or inhibiting nuclease activity of a RNP complex. The oligonucleotides and methods utilizing them may, by controlling the activity of CRISPR/Cas nucleases, may produce genetically engineered cells having fewer off-target mutations, fewer toxic effects on a biological system being modified (e.g., on a subject being treated or a cell or plurality of cells being genetically altered), and/or produce a higher number of genetically engineered cells with the modification than methods utilizing said oligosaccharides. Alternatively or in addition, the oligonucleotides and methods described herein may allow for sequential (multiplexed) editing of cells in which editing at a first target site sequence is performed and then inhibited/terminated, followed by editing at one or more additional target site sequences (e.g., using the same Cpf1 nuclease, or another RNA-guided nuclease (e.g., Cpf1, Cas9).

Accordingly, provided herein are genetically engineered cells, populations thereof, and cells descended therefrom, that are produced using methods described herein (e.g., using oligonucleotides described herein). Also provided are pharmaceutical compositions comprising said cell(s), e.g., and one or more pharmaceutically acceptable carriers and/or excipients.

Also provided herein are CRISPR/Cas systems or components thereof and optionally an oligonucleotide described herein. In some embodiments, a CRISPR/Cas system or components thereof are provided as a ribonucleoprotein (RNP) complex, e.g., comprising a CRISPR/Cas nuclease and a gRNA. In some embodiments, the RNP complex comprises an oligosaccharide described herein, e.g., an oligosaccharide specific to the gRNA or to another different gRNA. In some embodiments, CRISPR/Cas systems or components thereof are provided as one or more nucleic acids encoding a CRISPR/Cas nuclease, one or more gRNAs, and/or one or more oligonucleotides. In some embodiments, CRISPR/Cas systems or components thereof are provided as a system. For example, a system may comprise a CRISPR/Cas systems or components thereof in vitro, e.g., with a CRISPR/Cas nuclease and a gRNA in one or more containers and optionally an oligonucleotide (e.g., specific to the gRNA) in a separate container. In a further example, a system may be present in a biological system, e.g., a cell.

The compositions and methods provided herein may be applied to any cell or cell type capable of being genetically engineered using a CRISPR/Cas system as described herein. In some embodiments, the cell is a eukaryotic cell. In some embodiments, the cell is a mammalian cell, yeast cell, fungal cell, or plant cell. In some embodiments, the cell is a human cell or a mouse cell. In some embodiments, the cells may be obtained from a subject, such as a human subject. In some embodiments, the cells are obtained from a human subject, such as a human subject having a disease or disorder, such as a hematopoietic malignancy. In some embodiments, the cells are obtained from a healthy donor. Methods of obtaining mammalian cells, such as hematopoietic stem cells are described, e.g., in PCT Application No. PCT/US2016/057339, which is herein incorporated by reference in its entirety. In some embodiments, the mammalian subject is a non-human primate, a rodent (e.g., mouse or rat), a bovine, a porcine, an equine, or a domestic animal.

In some embodiments, the cells provided herein are stem cells. In some embodiments, the stem cells are embryonic stem cells, adult stem cells, induced pluripotent stem cells, cord blood stem cells, or amniotic fluid stem cells. In some embodiments, the stem cells are hematopoietic stem cells, mesenchymal stem cells, neural stem cells, epithelial stem cells, or skin stem cells. In some embodiments, the cells provided herein are progenitor cells, which are cells descended from a stem cell and capable to differentiate into a plurality of cell types.

In some embodiments, the cells provided herein are hematopoietic cells, e.g., hematopoietic stem cells (HSC) or hematopoietic progenitor cells (HPC). In some embodiments, the cells provided herein hematopoietic stem or progenitor cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage. In some embodiments, the HSCs are peripheral blood HSCs.

In some embodiments, the cells provided herein are immune effector cells. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell.

The skilled artisan will understand, however, that the provision of such examples is for the purpose of illustrating some specific embodiments, and additional suitable cells and cell types will be apparent to the skilled artisan based on the present disclosure, which is not limited in this respect.

In some embodiments, a genetically engineered cell provided herein comprises one genomic modification, e.g., a genomic modification that results in a loss of expression of a protein, for example a protein encoded by or regulated by the target site sequence, or expression of a variant form of the protein. It will be understood that the gene editing methods provided herein may result in genomic modifications in one or both alleles of a target genetic loci. In some embodiments, genetically engineered cells comprising a genomic modification in both alleles of a given genetic locus are preferred.

In some embodiments, a genetically engineered cell provided herein comprises two or more genomic modifications. For example, a population of genetically engineered cells can comprise a plurality of different mutations, such as two or more mutations in the same or different genetic loci in a cell.

As will be evident to one of ordinary skill in the art, the compositions and methods described herein may be used to modify any genetic locus in a cell, including for example protein-coding, non-protein coding, chromosomal, and extra-chromosomal sequences. Accordingly, targeting domains of gRNAs and corresponding sequences of oligonucleotides described herein may be designed to target any genetic locus (i.e., a target site sequence), such as a target site sequence adjacent to a PAM sequence for a corresponding CRISPR/Cas nuclease.

In some embodiments, the targeting domain targets a cell surface protein, such as a Type 0, Type 1, or Type 2 cell surface protein. In some embodiments, the targeting domain targets BCMA, CD19, CD20, CD30, ROR1, B7H6, B7H3, CD23, CD33, CD38, C-type lectin like molecule-1, CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and/or CD26. In some embodiments, a lineage-specific cell-surface antigen is chosen from: CD33, CD19, CD123, CLL-1 (CD371), CD30, CD5, CD6, CD7, CD38, and BCMA. In some embodiments, a lineage-specific cell-surface antigen is chosen from: CD7, CD13, CD19, CD22, CD25, CD32, CD33, CD38, CD44, CD47, CD56, 96, CD117, CD123, CD135, CD174, CLL-1, folate receptor b, IL1RAP, MUC1, NKG2D/NKG2DL, TIM-3, and WT1.

In some embodiments, the targeting domain targets a cell surface protein associated with a neoplastic or malignant disease or disorder, e.g., with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

Additional non-limiting examples of cell surface proteins include CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362, CD363, CD364, CD365, CD366, CD367, CD368, CD369, CD370, or CD371. See also examples of lineage-specific cell-surface antigens from BD Biosciences Human CD Marker Chart, bdbiosciences.com/content/dam/bdb/campaigns/reagent-education/BD_Reagents_CDMarkerHuman_Poster.pdf (incorporated by reference in its entirety).

Compositions and methods (e.g., exemplary gRNAs) for genetic editing and/or inhibition of genes encoding cell surface proteins (e.g., lineage specific antigens) are known to those of skill in the art and include, but are not limited to, those taught in PCT publications WO 2017/066760, WO 2020/047164A1, WO 2020/150478A1, WO 2020/237217A1, WO 2021/041971A1, and WO 2021/041977A1, which are incorporated by reference in their entirety. Additional compositions and methods (e.g., exemplary gRNAs) for genetic editing and/or inhibition of genes are known to those of skill in the art and include, but are not limited to, those taught in PCT publications WO 2017/186718A1 and WO 2018/083071A1, and in Mandal et al. Cell Stem Cell. (2014) 15(5): 643-52, which are incorporated by reference in their entirety.

### Methods of Using Oligonucleotides

Aspects of the present disclosure provide methods of producing a genetically engineered cell, comprising contacting the cell with one or more gRNAs (e.g., described herein) and a Cpf1 that binds the first gRNA, and contacting the cell with one or more oligonucleotides described herein. In some embodiments, contacting the cell with a gRNA and a Cpf1 nuclease forms a ribonucleoprotein complex comprising the gRNA and the Cpf1 nuclease, thus forming a RNP complex and allows the RNP complex to bind a target site sequence in the genome of the cell. In some embodiments, the cell may be contacted with more than one gRNA (e.g., gRNAs having different targeting sequences and/or crRNA sequences). For example, a cell may be contacted with a first gRNA having a first targeting domain sequence and second gRNA having a second targeting domain sequence. In some embodiments, the more than one gRNAs may target distinct genetic loci (e.g., different genes or regions of genes). In some embodiments, the cell may be contacted with more than one oligonucleotide (e.g., oligonucleotides having different sequence). For example, a cell may be contacted with a first oligonucleotide having a first sequence (e.g., corresponding to a first gRNA) and a second oligonucleotide having a second sequence (e.g., corresponding to a second gRNA). The disclosure is directed, in part, to the discovery that a first oligonucleotide can provide temporal/spatial control over CRISPR system activity on a first target sequence and/or with a first gRNA, and, e.g., a second oligonucleotide can provide temporal/spatial control over CRISPR system activity on a second target sequence and/or with a second gRNA.

In some embodiments, an RNP complex is formed, e.g., in vitro, and the cell is contacted with the RNP complex, e.g., via electroporation of the RNP complex into the cell. In some embodiments, the cell is contacted with CRISPR/Cas nuclease (e.g., Cpf1, Cas9) and gRNA separately, and the RNP complex is formed within the cell. In some embodiments, the cell is contacted with a nucleic acid, e.g., a DNA or RNA, encoding the CRISPR/Cas nuclease, and/or with a nucleic acid encoding the gRNA, or both. In some embodiments, the nucleic acid encoding the CRISPR/Cas protein and/or the nucleic acid encoding the gRNA is an mRNA or an mRNA analog. In some embodiments, the RNP complex and its components are formed, e.g., in vivo. In some embodiments, the cell is contacted with a nucleic acid encoding the CRISPR/Cas nuclease and a gRNA or nucleic acid encoding the gRNA, and the RNP complex forms within the cell (e.g., after expression of the CRISPR/Cas nuclease and optionally the gRNA).

Following contact of the cell with the gRNA and Cpf1 nuclease, or RNP complex thereof, the cell may be incubated allowing for Cpf1 nuclease activity to occur, e.g., binding and cleavage of at least one strand of DNA at the target site sequence in the genome of the cell. Cleavage by the Cpf1 nuclease may be reduced or terminated at a desired time point by contacting the cell with an oligonucleotide described herein. In some embodiments, contacting the cell with the oligonucleotide reduces or eliminates the cleavage by the Cpf1 nuclease at the target site sequence is reduced or eliminated.

In some embodiments, contacting the cell with the oligonucleotide results in at least a 10%, 15%, 20%, 25%, 30%, 35%, 40%, 45%, 50%, 55%, 60%, 65%, 70%, 75%, 80%, 85%, 90%, 95%, or up to 100% reduction of cleavage by the Cpf1 nuclease at the target site sequence. Methods of assessing functionality of a gRNA and cleavage by a Cpf1 nuclease at a target site sequence may be performed for example using methods known in the art.

In some embodiments, contacting the cell with an oligonucleotide results in at least a 10%, 15%, 20%, 25%, 30%, 35%, 40%, 45%, 50%, 55%, 60%, 65%, 70%, 75%, 80%, 85%, 90%, 95%, or up to 100% reduction of at least one gRNA activity. Any activity or function of a gRNA may be assessed to evaluate efficacy of the oligonucleotide, e.g., binding to a Cpf1 nuclease, recruiting a Cpf1 nuclease to the target site sequence, directing cleavage of the target site sequence.

In some embodiments, the disclosure is directed to a method of producing a genetically engineered cell, comprising contacting a cell with a gRNA (e.g., a first gRNA) and a Cpf1 nuclease and contacting the cell with an oligonucleotide, where the oligonucleotide comprises a first region complementary to a targeting domain of the gRNA and a second region complementary to a crRNA sequence of the gRNA. In some embodiments, contacting the cell with the oligonucleotide reduces genomic editing by the Cpf1 nuclease at the target sequence of the gRNA. In some embodiments, contacting the cell with the oligonucleotide reduces or eliminates interaction between one, two, or all of: the gRNA and the Cpf1 nuclease; the gRNA and its target sequence (e.g., in the genome of the cell); or an RNP complex comprising the gRNA and Cpf1 nuclease and the target sequence. In some embodiments, contacting the cell with the oligonucleotide inhibits formation or maintenance of a RNP complex comprising the gRNA and the Cpf1 nuclease. In some embodiments, contacting the cell with the oligonucleotide inhibits nuclease activity of a RNP complex comprising the gRNA and the Cpf1 nuclease and/or reduces interaction between the RNP complex and a target sequence in the genome of the cell.

In some embodiments, contacting a cell comprising a Cpf1 nuclease and a gRNA with an oligonucleotide (comprising a first region complementary to a targeting domain of the gRNA and a second region complementary to a crRNA sequence of the gRNA) decreases or eliminates Cpf1 nuclease activity with respect to the target sequence of the gRNA or the particular gRNA. The oligonucleotides provided herein may thus provide temporal and/or spatial control over the activity a CRISPR/Cas system. In some embodiments, a cell is contacted with components of a CRISPR system (e.g., a gRNA comprising a targeting domain complementary to a target sequence and a Cpf1 nuclease) and simultaneously or in temporal proximity contacted with an oligonucleotide (e.g., specific to the gRNA and/or target sequence). As used herein, temporal proximity refers to a nearness of two events in time. In some embodiments, a later event that occurs in temporal proximity to an earlier event occurs less than or equal to 1, 2, 3, 4, 5, 6, 7, 8, 9, 10, 15, 20, 30, 40, 50, 60, 90, or 120 minutes after the earlier event. In some embodiments, an earlier event that occurs in temporal proximity to a later event occurs less than or equal to 1, 2, 3, 4, 5, 6, 7, 8, 9, 10, 15, 20, 30, 40, 50, 60, 90, or 120 minutes before the later event. In some embodiments, a cell is contacted with components of a CRISPR system (e.g., a gRNA comprising a targeting domain complementary to a target sequence and a CRISPR/Cas nuclease (e.g., a Cpf1 nuclease)) and contacted with an oligonucleotide (e.g., specific to the gRNA and/or target sequence) within 1, 2, 3, 4, 5, 6, 7, 8, 9, 10, 11, 12, 18, or 24 hours (and optionally after at least 30 minutes, or after at least 1, 2, 3, or 4 hours). Without wishing to be bound by theory, the disclosure is directed in part to methods where a cell is contacted with the components of a CRISPR system (e.g., a gRNA comprising a targeting domain complementary to a target sequence and a Cpf1 nuclease), and contacted with an oligonucleotide (e.g., specific to the gRNA and/or target sequence) such that the CRISPR system has sufficient time to genetically modify a target in the genome of the cell and thereafter is expeditiously inactivated to avoid undesired effects such as off-target activity or toxicity.

Such control can be applied to CRISPR systems not comprising nuclease activity, e.g., comprising dead CRISPR/Cas nucleases (e.g., a dead Cpf1 nuclease lacking nuclease activity). In some embodiments, a dead CRISPR/Cas nuclease comprises one or more functional domains, e.g., a base editor domain. In some embodiments, contacting a cell with an oligonucleotide reduces or inactivates the functional domain activity (e.g., base editing activity) of an RNP complex comprising a dead CRISPR/Cas nuclease and a gRNA, where the oligonucleotide is specific to the gRNA or the target sequence of the gRNA.

Also within the scope of the present disclosure are cells containing more than one oligonucleotide, e.g., an oligonucleotide for each gRNA present in the cell, e.g., wherein the gRNAs targets different genetic loci. Accordingly, the present disclosure is also directed, in part, to methods comprising contacting a cell with a first oligonucleotide and contacting the cell with a second oligonucleotide, where each oligonucleotide is specific to a different gRNA and/or different target sequence.

In some embodiments, a method described herein comprises contacting a cell with a second gRNA comprising a second targeting domain capable of binding a second target sequence (e.g., after contacting the cell with a first gRNA and CRISPR/Cas nuclease). In some embodiments, the second gRNA is compatible with the CRISPR/Cas nuclease previously added to the cell (e.g., with a Cpf1 nuclease previously added to the cell), e.g., comprises a crRNA sequence compatible with the CRISPR/Cas nuclease previously added. In some embodiments, the second gRNA is not compatible with the CRISPR/Cas nuclease previously added to the cell (e.g., with a Cpf1 nuclease previously added to the cell), e.g., comprises a crRNA sequence not compatible with the CRISPR/Cas nuclease previously added. In some embodiments, the second gRNA comprises a crRNA sequence compatible with a different CRISPR/Cas nuclease.

In some embodiments, a method described herein comprises contacting a cell with a second CRISPR/Cas nuclease that binds to the second gRNA, forming a ribonucleoprotein (RNP) complex under conditions suitable for the second gRNA to form and/or maintain an RNP complex with the second CRISPR/Cas nuclease and for the RNP complex to bind a second target sequence in the genome of the cell. In some embodiments, the second CRISPR/Cas nuclease is a Cpf1 nuclease (e.g., the same Cpf1 nuclease as the first CRISPR/Cas nuclease, or a different Cpf1 nuclease than the first CRISPR/Cas nuclease). In some embodiments, the second CRISPR/Cas nuclease is not a Cpf1 nuclease.

In some embodiments, contacting the cell with a second gRNA and contacting the cell with a second CRISPR/Cas nuclease that binds to the second gRNA occurs simultaneously or in temporal proximity to one another. In some embodiments, said contacting steps comprise contacting the cell with an RNP comprising the second gRNA and second CRISPR/Cas nuclease. In some embodiments, contacting the cell with a second gRNA (and optionally with a second CRISPR/Cas nuclease that binds to the second gRNA) occurs less than or equal to 1, 2, 3, 4, 5, 6, 7, 8, 9, 10, 15, 20, 30, 40, 50, 60, 90, or 120 minutes after, or less than or equal to 1, 2, 3, 4, 5, 6, 7, 8, 9, 10, 11, 12, 18, or 24 hours after, or less than or equal to 2, 3, 4, 5, 6, 7, 8, 9, 10, 11, 12, 13, or 14 days after contacting the cell with a first gRNA and first CRISPR/Cas nuclease.

The disclosure contemplates methods comprising all possible temporal arrangements of the steps described herein. In some embodiments, contacting the cell with an oligonucleotide specific to the first gRNA and/or first target sequence occurs simultaneously or in temporal proximity to contacting the cell with a second gRNA (and optionally with contacting the cell with a second CRISPR/Cas nuclease). In some embodiments, contacting the cell with an oligonucleotide specific to the first gRNA and/or first target sequence occurs before contacting the cell with a second gRNA (and optionally contacting the cell with a second CRISPR/Cas nuclease), e.g., less than or equal to 1, 2, 3, 4, 5, 6, 7, 8, 9, 10, 15, 20, 30, 40, 50, 60, 90, or 120 minutes before, or less than or equal to 1, 2, 3, 4, 5, 6, 7, 8, 9, 10, 11, 12, 18, or 24 hours before (and optionally at least 30 seconds or 1 minute before). In some embodiments, contacting the cell with an oligonucleotide specific to the first gRNA and/or first target sequence occurs after contacting the cell with a second gRNA (and optionally contacting the cell with a second CRISPR/Cas nuclease), e.g., less than or equal to 1, 2, 3, 4, 5, 6, 7, 8, 9, 10, 15, 20, 30, 40, 50, 60, 90, or 120 minutes after (and optionally at least 30 seconds or 1 minute after).

In some embodiments, the method further comprises contacting the cell with a second oligonucleotide that reduces genomic editing at the second target sequence. In some embodiments, the second oligonucleotide is specific to the second gRNA and/or the second target sequence. In some embodiments, contacting the cell with the second oligonucleotide reduces genomic editing by the CRISPR/Cas nuclease at the second target sequence of the second gRNA. In some embodiments, contacting the cell with the second oligonucleotide reduces or eliminates interaction between one, two, or all of: the second gRNA and a CRISPR/Cas nuclease (e.g., a Cpf1 nuclease); the second gRNA and its target sequence (e.g., in the genome of the cell); or an RNP complex comprising the second gRNA and a CRISPR/Cas nuclease (e.g., a Cpf1 nuclease) and the second target sequence. In some embodiments, contacting the cell with the second oligonucleotide inhibits formation or maintenance of a RNP complex comprising the second gRNA and a CRISPR/Cas nuclease (e.g., a Cpf1 nuclease). In some embodiments, contacting the cell with the second oligonucleotide inhibits nuclease activity of a RNP complex comprising the second gRNA and a CRISPR/Cas nuclease (e.g., a Cpf1 nuclease) and/or reduces interaction between the RNP complex and the second target sequence in the genome of the cell.

In some embodiments, contacting the cell with a second oligonucleotide specific to a second gRNA and/or second target sequence occurs simultaneously or in temporal proximity to contacting the cell with a second gRNA (and optionally with contacting the cell with a second CRISPR/Cas nuclease). In some embodiments, contacting the cell with an oligonucleotide specific to the first gRNA and/or first target sequence occurs after contacting the cell with a second gRNA (and optionally contacting the cell with a second CRISPR/Cas nuclease), e.g., less than or equal to 1, 2, 3, 4, 5, 6, 7, 8, 9, 10, 15, 20, 30, 40, 50, 60, 90, or 120 minutes after, or less than or equal to 1, 2, 3, 4, 5, 6, 7, 8, 9, 10, 11, 12, 18, or 24 hours after (and optionally at least 30 seconds or 1 minute after).

The disclosure is directed, in part, to the discovery that an oligonucleotide described herein may be specific to a gRNA or target sequence, and consequently only affect complexes comprising said gRNA or CRISPR/Cas nuclease associated activity aimed at said target sequence (e.g., and not complexes comprising a different gRNA or CRISPR/Cas nuclease associated activity aimed at a different target sequence). For example, a method described herein may comprise contacting a cell (in a plurality of steps) with a first gRNA (e.g., comprising a targeting domain complementary to a first target sequence), a first oligonucleotide specific to the first gRNA or the first target sequence, a second gRNA (e.g., comprising a targeting domain complementary to a second target sequence), and a second oligonucleotide specific to the second gRNA or second target sequence. In some embodiments, the first oligonucleotide does not substantially bind (e.g., does not bind) 1, 2, 3, or any of: the second gRNA, an RNP complex comprising a CRISPR/Cas nuclease and the second gRNA, a CRISPR/Cas nuclease included with the second gRNA, or the second target sequence. In some embodiments, the first oligonucleotide does not substantially inhibit (e.g., does not inhibit) formation or maintenance of a RNP complex comprising the second gRNA and a CRISPR/Cas nuclease (e.g., a Cpf1 nuclease). In some embodiments, the first oligonucleotide does not substantially inhibit the ability of a RNP complex comprising the second gRNA and a CRISPR/Cas nuclease (e.g., Cpf1 nuclease) to bind the second target sequence in the genome of a cell. In some embodiments, the first oligonucleotide does not substantially inhibit nuclease activity of a RNP complex comprising the second gRNA and a CRISPR/Cas nuclease (e.g., Cpf1 nuclease), or said complex's ability to bind the second target sequence in the genome of a cell. In some embodiments, the second oligonucleotide does not substantially bind (e.g., does not bind) 1, 2, 3, or any of: the first gRNA, an RNP complex comprising a CRISPR/Cas nuclease and the first gRNA, a CRISPR/Cas nuclease included with the first gRNA, or the first target sequence. In some embodiments, the second oligonucleotide does not substantially inhibit (e.g., does not inhibit) formation or maintenance of a RNP complex comprising the first gRNA and a CRISPR/Cas nuclease (e.g., a Cpf1 nuclease). In some embodiments, the second oligonucleotide does not substantially inhibit the ability of a RNP complex comprising the first gRNA and a CRISPR/Cas nuclease (e.g., Cpf1 nuclease) to bind the first target sequence in the genome of a cell. In some embodiments, the second oligonucleotide does not substantially inhibit nuclease activity of a RNP complex comprising the first gRNA and a CRISPR/Cas nuclease (e.g., Cpf1 nuclease), or said complex's ability to bind the first target sequence in the genome of a cell.

As used herein, “does not substantially” in the context of an agent's effect on an action or phenomenon, such as binding or nuclease activity, refers to the absence of a significant effect on the action or phenomenon by the subject. In some embodiments, an agent that does not substantially bind to another agent does not detectably bind under relevant conditions (e.g., cellular conditions) or as assessed by a binding assay. In some embodiments, an agent that does not substantially inhibit an activity or phenomenon decreases the occurrence or level of the activity or phenomenon by less than 50, 40, 30, 20, 10, 5, or 1%.

The disclosure is directed, in part, to the discovery that an oligonucleotide described herein may be specific to a CRISPR/Cas nuclease or class of nucleases (e.g., Cpf1 nucleases), and consequently only affect complexes comprising the compatible CRISPR/Cas nuclease or a member of the class of nucleases (e.g., a Cpf1 nuclease) (e.g., and not complexes comprising a different CRISPR/Cas nuclease (e.g., a Cas9 nuclease). For example, a method described herein may comprise contacting a cell (in a plurality of steps) with a first gRNA (e.g., comprising a targeting domain complementary to a first target sequence) and a first CRISPR/Cas nuclease (e.g., a first Cpf1 nuclease), an oligonucleotide specific to the first gRNA or the first target sequence and the first CRISPR/Cas nuclease, and a second gRNA (e.g., comprising a targeting domain complementary to a second target sequence) and a second CRISPR/Cas nuclease (e.g., a Cas9 nuclease). In some embodiments, the oligonucleotide does not substantially bind (e.g., does not bind) 1, 2, 3, or any of: the second gRNA, an RNP complex comprising the second CRISPR/Cas nuclease and the second gRNA, the second CRISPR/Cas nuclease included with the second gRNA, or the second target sequence. In some embodiments, the oligonucleotide does not substantially inhibit (e.g., does not inhibit) formation or maintenance of a RNP complex comprising the second gRNA and the second CRISPR/Cas nuclease (e.g., a Cas9 nuclease). In some embodiments, the oligonucleotide does not substantially inhibit the ability of a RNP complex comprising the second gRNA and the second CRISPR/Cas nuclease (e.g., Cas9 nuclease) to bind the second target sequence in the genome of a cell. In some embodiments, the oligonucleotide does not substantially inhibit nuclease activity of a RNP complex comprising the second gRNA and the second CRISPR/Cas nuclease (e.g., Cas9 nuclease), or said complex's ability to bind the second target sequence in the genome of a cell.

The disclosure further contemplates methods for producing a genetically engineered cell comprising multiple editing steps and inhibition steps, allowing the simultaneous or in temporal proximity editing of multiple genomic cites while selectively inhibiting one, a group of, or all CRISPR/Cas nucleases after editing has been accomplished.

A method of the disclosure may comprise contacting a cell with n different gRNAs, where n is an integer ≥2, wherein each of the n different gRNAs comprise a targeting domain complementary to a target sequence. In some embodiments, n is at least 2, 3, 4, 5, 6, 7, 8, 9, 10, 11, 12, 13, 14, 15, 16, 17, 18, 19, or 20 (and optionally, no more than 30, 25, 20, 15, or 10). In some embodiments, each of the n different gRNAs comprises a targeting domain complementary to a different target sequence. In some embodiments, the n different gRNAs comprise targeting domains complementary to n−1 or fewer different target sequences (and optionally at least 2 different target sequences). In some embodiments, such a method may comprise contacting a cell (in one or a plurality of steps) with up to n different oligonucleotides, each specific to a different gRNA of the n different gRNAs. The disclosure encompasses methods that provide temporal and/or spatial control over n gRNAs and/or RNPs comprising said gRNAs, and/or the activity of CRISPR/Cas nucleases at up to n different target sequences.

### Methods of Administration to Subjects in Need Thereof

The disclosure is also directed, in part, to methods of administering to a subject in need thereof a composition described herein, e.g., a cell genetically engineered via a method described herein, a population or descendent thereof, or a pharmaceutical composition comprising the same. The cell, population of cells, or descendants thereof may comprise one or more modifications (e.g., genetic modifications) relative to a wildtype cell. In some embodiments, the cell, population of cells, or descendants thereof comprise a modification to a first gene relative to a wildtype cell of the same type. In some embodiments, the cell, population of cells, or descendants thereof comprise a modification to a second gene relative to a wildtype cell of the same type. Genes modified may correspond to any genetic locus targetable by a method described herein, e.g., a gene encoding a cell surface protein described herein.

In some embodiments, the methods further involve administering to the subject a therapeutically effective amount of at least one agent that targets a product encoded by a wildtype copy of the modified gene. Without wishing to be bound by theory, by administering an agent that targets a product encoded by a wildtype copy of the modified gene in combination with a cell, population of cells, or descendants thereof comprising the modified gene, it is possible to target cells within a subject with the agent (e.g., disease cells, e.g., cancer cells) while not targeting or targeting to a lesser degree the cell, population of cells, or descendants thereof. For example, such a method may be used to selectively ablate or kill a target cell population in a subject while in combination replenishing the subject with new cells not vulnerable to the agent. As a further example, such a method may administer the agent as a part of the cell, population of cells, or descendants thereof (e.g., a CAR-T therapeutic), and would thus avoid or decrease cell fratricide. In some embodiments, administration of the at least one agent targeting the product encoded by the wildtype copy of the modified gene occurs simultaneously or in temporal proximity with administration of the cell, population or descendant thereof, or the pharmaceutical composition. In some embodiments, administration of the at least one agent targeting the product encoded by the wildtype copy of the modified gene occurs after administration of the cell, population or descendant thereof, or the pharmaceutical composition. In some embodiments, administration of the at least one agent targeting the product encoded by the wildtype copy of the modified gene occurs before administration of the cell, population or descendant thereof, or the pharmaceutical composition. In some embodiments, where the cell, population of cells, or descendants thereof comprises a modification to a first gene and a second gene relative to a wildtype cell of the same type, the method may comprise administering one or more (e.g., two agents) targeting the products of the first gene and the second gene (e.g., wildtype copies of the first gene and the second gene).

A subject in need thereof is, in some embodiments, a subject undergoing or about to undergo therapy involving administration with the agent, such as an immunotherapeutic, targeting a product of the first gene and/or second gene. A subject in need thereof is, in some embodiments, a subject having or having been diagnosed with, a malignancy, such as cancer (e.g., cancer associated with the presence of cancer stem cells, a hematopoietic malignancy). In some embodiments, a subject having such a malignancy may be a candidate for agent, such as an immunotherapeutic, targeting a product of the first gene and/or second gene, but the risk of detrimental on-target, off-disease effects may outweigh the benefit, expected or observed, to the subject.

In some such embodiments, administration of genetically engineered cells as described herein, results in an amelioration of the detrimental on-target, off-disease effects, as the genetically engineered cells provided herein are not targeted efficiently by the agent, such as an immunotherapeutic, targeting a product of the first gene and/or second gene.

In some embodiments, the malignancy is a hematologic malignancy, or a cancer of the blood. In some embodiments, the malignancy is a lymphoid malignancy or a myeloid malignancy.

In some embodiments, the malignancy is an autoimmune disease or disorder. Examples of autoimmune disorders include, without limitation, rheumatoid arthritis, multiple sclerosis, leukemia, graft-versus host disease, lupus, and psoriasis.

Also within the scope of the present disclosure are malignancies that are considered to be relapsed and/or refractory, such as relapsed or refractory hematological malignancies. A subject in need thereof is, in some embodiments, a subject undergoing or that will undergo an immune effector cell therapy targeting a product of the first gene and/or second gene, e.g., CAR-T cell therapy, wherein the immune effector cells express a CAR targeting the product, and wherein at least a subset of the immune effector cells also express the product on their cell surface. As used herein, the term “fratricide” refers to self-killing. For example, cells of a population of cells kill or induce killing of cells of the same population. In some embodiments, cells of the immune effector cell therapy kill or induce killing of other cells of the immune effector cell therapy.

In such embodiments, fratricide ablates a portion of or the entire population of immune effector cells before a desired clinical outcome, e.g., ablation of malignant cells expressing the product within the subject, can be achieved. In some such embodiments, using genetically engineered immune effector cells, as provided herein, e.g., immune effector cells that do not express the product or do not express a variant of the product recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express the product or do not express a variant of the product recognized by the CAR, may be further modified to also express the agent (e.g., a CAR targeting the product). In some embodiments, the immune effector cells may be lymphocytes, e.g., T-lymphocytes, such as, for example alpha/beta T lymphocytes, gamma/delta T-lymphocytes, or natural killer T cells. In some embodiments, the immune effect or cells may be natural killer (NK) cells.

In some embodiments, an effective number of genetically engineered cells as described herein, comprising a modification in their genome is administered to a subject in need thereof, e.g., a subject undergoing or that will undergo a therapy targeting a product of the first gene and/or second gene, wherein the therapy is associated or is at risk of being associated with a detrimental on-target, off-disease effect, e.g., in the form of cytotoxicity towards healthy cells in the subject that express the product. In some embodiments, an effective number of such genetically engineered cells may be administered to the subject in combination with the agent targeting a product encoded by a first gene or a second gene.

It is understood that when genetically modified cells and agents targeting a product encoded by a first gene or a second gene (e.g., an immunotherapeutic agent) are administered in combination, the cells and the agent may be administered at the same time or at different times, e.g., in temporal proximity.

For example, in some embodiments, administration in combination includes administration in the same course of treatment, e.g., in the course of treating a subject with an agent targeting a product (e.g., immunotherapy), the subject may be administered an effective number of genetically engineered cells, simultaneously, concurrently, or sequentially, e.g., before, during, or after the treatment with the agent, and/or in any order with respect to each other and the cells, population of cells, or descendants thereof. Furthermore, the cells and the agent may be admixed or in separate volumes or dosage forms.

In some embodiments, the agent that targets a product encoded by the first gene or a wildtype copy thereof is an immunotherapeutic agent. In some embodiments, the agent that targets a product encoded by the first gene or a wild-type copy thereof comprises an antigen-binding fragment that binds the product encoded by the first gene or a wildtype copy thereof.

In some embodiments, the agent is an immune cell that expresses a chimeric antigen receptor, which comprises an antigen-binding fragment (e.g., a single-chain antibody) capable of binding to the product encoded by the first gene or a wild-type copy thereof. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise a CD6-binding antibody fragment. The antibody fragment can comprise one or more CDRs, the variable regions (or portions thereof), the constant regions (or portions thereof), or combinations of any of the foregoing.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in PCT Publication No. WO 2019/178382, and in Table C below.

TABLE C
Exemplary components of a chimeric receptor

| Chimeric receptor component | Amino acid sequence |
|---|---|
| Antigen-binding fragment | Light chain- Linker-Heavy chain |
| CD28 costimulatory domain | IEVMYPPPYLDNEKSNGTIIHVKGKHLCP |
| | SPLFPGPSKPFWVLVVVGGVLACYSLLVTV |
| | AFIIFWVRSKRSRLLHSDYMNMTPRRPGPT |
| | RKHYQPYAPPRDFAAYRS (SEQ ID NO: 7) |
| CD8alpha transmembrane | IYIWAPLAGTCGVLLLSLVITLYC |
| domain | (SEQ ID NO: 8) |
| CD28 transmembrane domain | FWVLVVVGGVLACYSLLVTVAFII |
| | FWVRSKRSRLLHSDYMNMTPRR |
| | PGPTRKHYQPYAPPRDFAAYRS |
| | (SEQ ID NO: 9) |
| 4-1BB intracellular domain | KRGRKKLLYIFKQPFMRVQTTQEEDGCS |
| | CRFPEEEEGGCEL (SEQ ID NO: 10) |
| CD33 cytoplasmic signaling | RVKFSRSADAPAYQQGQNQLYNELNLG |
| domain | RREEYDVLDKRRGRDPEMGGKPQRRKNP |
| | QEGLYNELQKDKMAEAYSEIGMKGERRR |
| | GKGHDGLYQGLSTATKDTYDALHMQALPPR |
| | (SEQ ID NO: 11) |

In some embodiments, the number of genetically engineered cells provided herein or immune effector cells (e.g., CAR-expressing cells) that are administered to a subject in need thereof, is within the range of 10⁶-10¹¹. However, amounts below or above this exemplary range are also within the scope of the present disclosure. For example, in some embodiments, the number of genetically engineered cells provided herein or immune effector cells (e.g., CAR-expressing cells) that are administered to a subject in need thereof is about 10⁶, about 10⁷, about 10⁸, about 10⁹, about 10¹⁰, or about 10¹¹. In some embodiments, the number of genetically engineered cells provided herein or immune effector cells (e.g., CAR-expressing cells) that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

In some embodiments, the agent that targets a product encoded by the first gene or a wildtype copy thereof is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the cell surface (e.g., target cell), thereby resulting in death of the target cell.

Toxins or drugs compatible for use in antibody-drug conjugates are known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018) 11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BJIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the product (target cells). The type of toxin or drug used in the antibody-drug conjugates described herein is not limited to any specific type.

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Example 1: Inhibition by Oligonucleotides and Effects on Cell Growth and Viability

This example demonstrates use of exemplary oligonucleotides of the disclosure in a method of producing a genetic modification of an exemplary cell type Cpf1. The example shows that oligonucleotides of the disclosure can rapidly eliminate on-target editing by the Cpf1 nuclease, and measures effects of oligonucleotides on viability and cell growth.

As shown in FIG. 1, CD34+ cells (hCD34+ HSPCs GCSF mobilized PB from HemaCare) were thawed, and 48 hours approximately 1×10⁶ cells were electroporated with ribonucleoprotein (RNP) complexes comprising Cpf1 nuclease from Acidaminococcus sp. BV3L6 (Alt-R Cas12a obtained from IDT) and a gRNA targeting a portion of the gene encoding CCR5 comprising a targeting sequence provided by SEQ ID NO: 3 (see, Table B) or gRNA targeting a portion of the gene encoding CD19 comprising a targeting sequence provided by SEQ ID NO: 4 (see, Table B). The electroporation was performed with an inhibitor oligonucleotide (+Inhibitor) that was specific for the target gRNA or in the absence of an inhibitor oligonucleotide (−Inhibitor). For example, electroporation of cells with RNP complexes comprising a gRNA targeting CCR5 and Cpf1 was performed in the presence or absence of a CCR5 inhibitor oligonucleotide comprising a first region that is complementary to the CCR5 targeting domain of the gRNA and a second region that is complementary to the crRNA sequence of the gRNA, see e.g., SEQ ID NO: 1 in Table A. Similarly, electroporation of cells with RNP complexes comprising a gRNA targeting CD19 and Cpf1 was performed in the presence or absence of a CD19 inhibitor oligonucleotide comprising a first region that is complementary to the CD19 targeting domain of the gRNA and a second region that is complementary to the crRNA sequence of the gRNA, see e.g., SEQ ID NO: 2 in Table A.

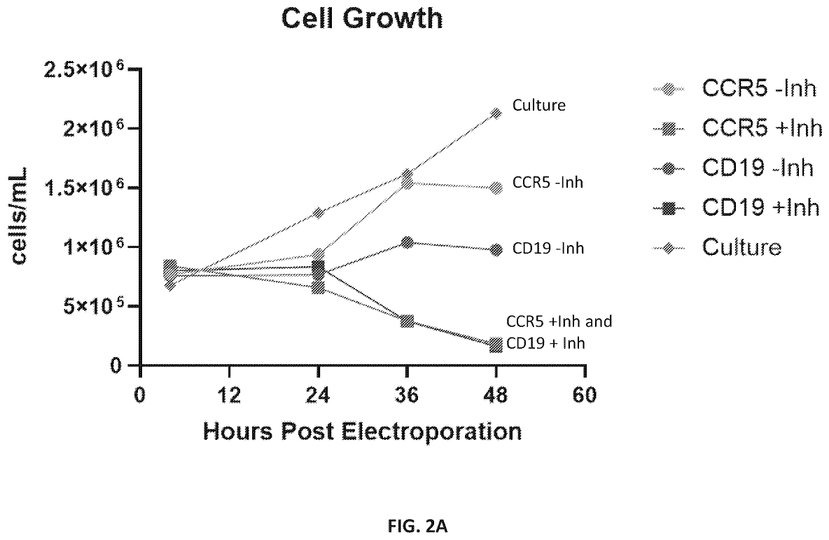

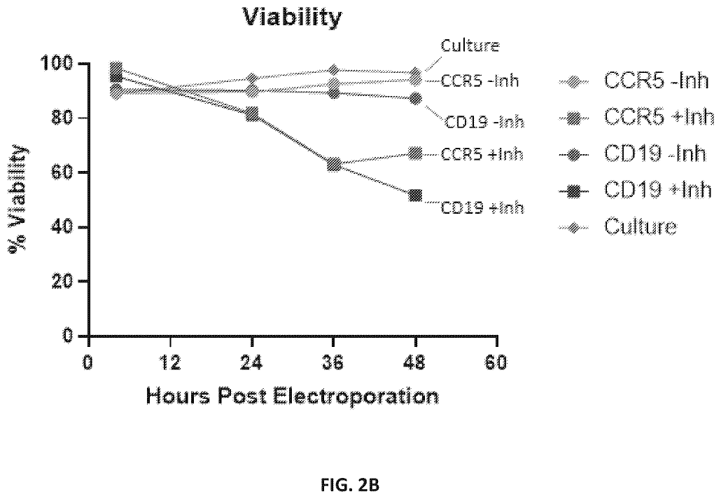

Cell growth rate and viability is presented in FIGS. 2A-2B. These data indicate that cells treated with the oligonucleotides showed decreased growth rate and viability over 48 hours post-electroporation.

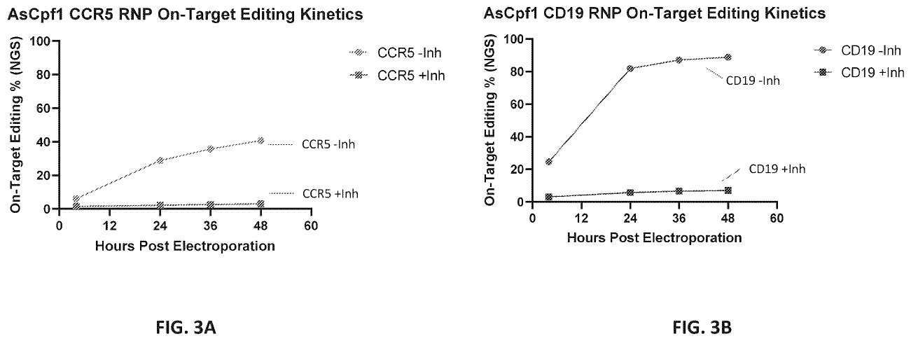

DNA samples were also extracted from the cells at the indicated time points. Next generation sequencing (NGS) was used to assess on-target editing by the Cpf1 nuclease and the gRNA at the CCR5 or CD19 target sites. As shown in FIGS. 3A and 3B, cells that were electroporated with Cpf1 and a gRNA targeting CCR5, or cells that were electroporated with Cpf1 and a gRNA targeting CD19 resulted in an increase in editing at the target site over the 48 hours following electroporation. In contrast, cells that were electroporated with Cpf1, a gRNA, and a corresponding oligonucleotide saw very low, if any, editing at the target site. See, FIGS. 3A and 3B. This data demonstrates a dramatic inhibition of on-target editing by target domain specific oligonucleotides.

### Example 2: Time Dependence of Oligonucleotide Inhibition

This example demonstrates time dependence of inhibition in the use of exemplary oligonucleotides of the disclosure in a method of producing a genetic modification of an exemplary CD34+ cell using exemplary CRISPR/Cas nuclease (AsCpf1). The example shows that cells must be contacted with oligonucleotides of the disclosure simultaneously or within a short time after contact with AsCpf1 to inhibit on-target editing. Effects of oligonucleotides on viability and cell growth at different time points were also measured.

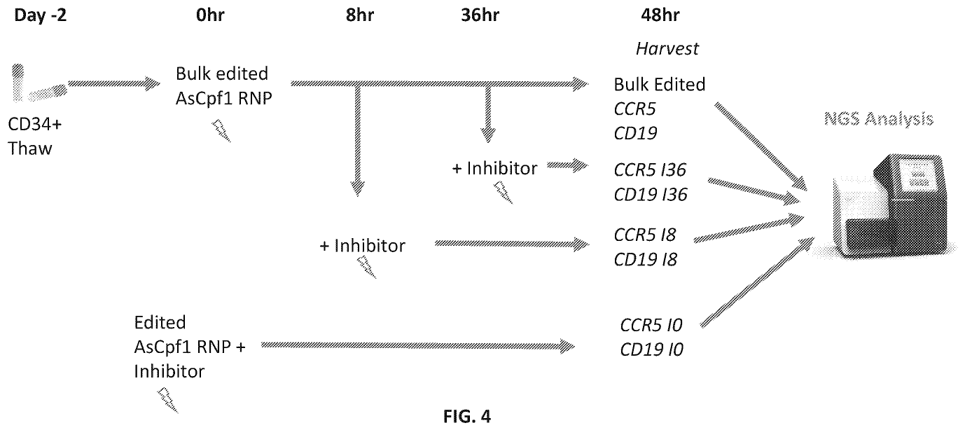

As shown in FIG. 4, CD34+ cells (hCD34+ HSPCs GCSF mobilized PB from HemaCare) were thawed, and 2 days later cells were electroporated with ribonucleoprotein (RNP) complexes comprising Cpf1 nuclease from Acidaminococcus sp. BV3L6 (Alt-R Cas12a obtained from IDT) and a gRNA targeting a portion of the gene encoding CCR5 comprising a targeting sequence provided by SEQ ID NO: 3 (see, Table B) or gRNA targeting a portion of the gene encoding CD19 comprising a targeting sequence provided by SEQ ID NO: 4 (see, Table B). A population of cells were electroporated with RNP complexes comprising a gRNA and Cpf1 nuclease in the presence of inhibitor oligonucleotides at time 0 (corresponding to CCR5 I0 and CD19 I0). The remaining cells were bulk edited by electroporating the cells with RNP complexes comprising a gRNA and Cpf1 nuclease. For these cells, the inhibitor oligonucleotides were electroporated at either 8 hours following the first electroporation (corresponding to CCR5 I8 and CD19 I8) or 36 hours following the first electroporation (corresponding to CCR5 I36 and CD19 I36).

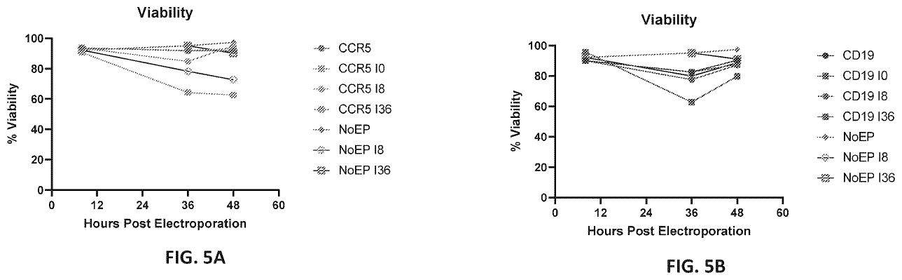

Cell viability was assessed at times post-electroporation over the next 48 hours (FIGS. 5A and 5B).

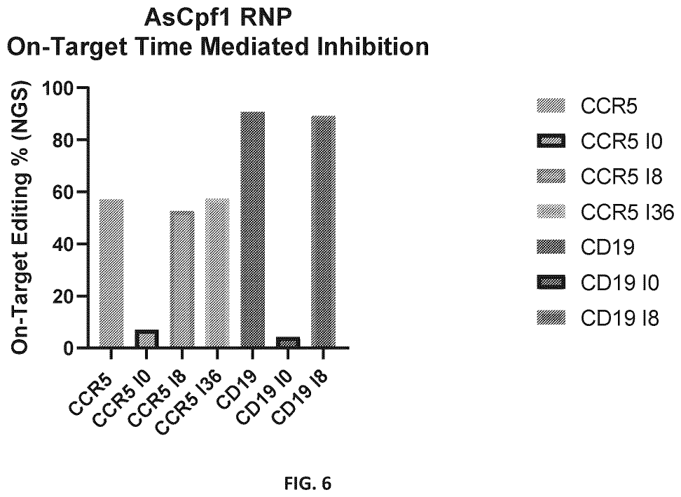

FIG. 6 shows on-target editing by Next Generation Sequencing (NGS) analysis at the indicated time points following electroporation. The cells that were electroporated with inhibitor oligonucleotides at the same time as the RNP complexes resulted in very little on-target editing, indicating that the oligonucleotides inhibited editing by the RNP complexes.

### Example 3: Target- and Nuclease-Dependent Oligonucleotide Inhibition

This example demonstrates that inhibition of genetic modification by the use of exemplary oligonucleotides of the disclosure is target-specific, whereas effects of oligonucleotides on cell growth and viability are general across the oligonucleotides tested.

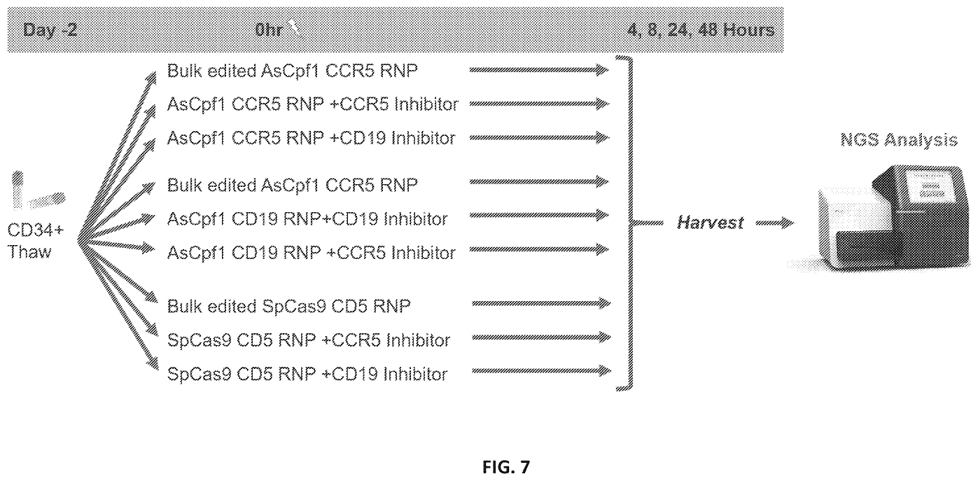

CD34+ cells were thawed as described in Examples 1 and 2 and electroporated with ribonucleoprotein (RNP) complexes comprising Cpf1 nuclease from Acidaminococcus sp. BV3L6 (Alt-R Cas12a obtained from IDT) and a gRNA targeting a portion of the gene encoding CCR5 comprising a targeting sequence provided by SEQ ID NO: 3 (see, Table B) or gRNA targeting a portion of the gene encoding CD19 comprising a targeting sequence provided by SEQ ID NO: 4 (see, Table B). Cells were co-electroporated inhibitor oligonucleotides at time 0 that targeted either the same gRNA targeting sequence or a different targeting sequence. For example, cells that were electroporated with RNP complexes comprising a gRNA targeting CCR5 were co-electroporated with oligonucleotides directed to the CCR5 gRNA or a different gRNA (i.e., the CD19 gRNA). Similarly, cells that were electroporated with RNP complexes comprising a gRNA targeting CD19 were co-electroporated with oligonucleotides directed to the CD19 gRNA or a different gRNA (i.e., the CCR5 gRNA). Additionally, other populations of cells were electroporated with ribonucleoprotein (RNP) complexes comprising Cas9 nuclease from Streptococcus pyogenes (obtained from Integrated DNA Technologies) and a gRNA targeting a portion of the gene encoding CD5 comprising a targeting sequence provided by SEQ ID NO: 5, either in the absence or presence of inhibitor oligonucleotides directed to the CD19 gRNA or CCR5 gRNA (FIG. 7).

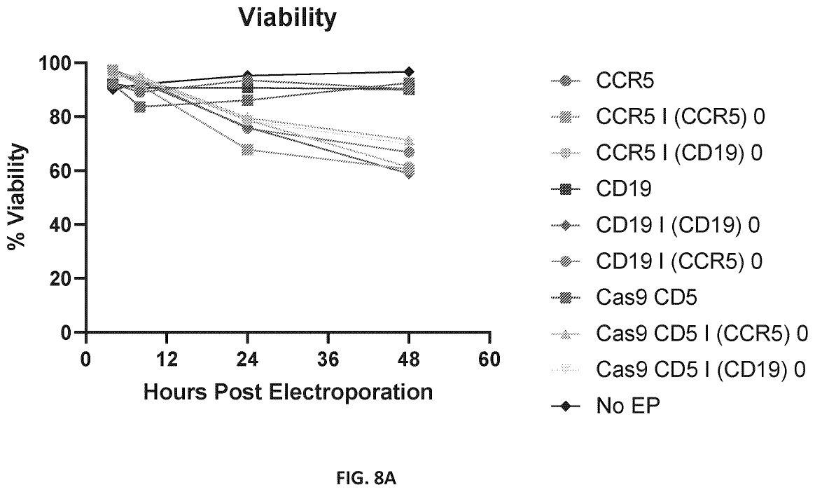

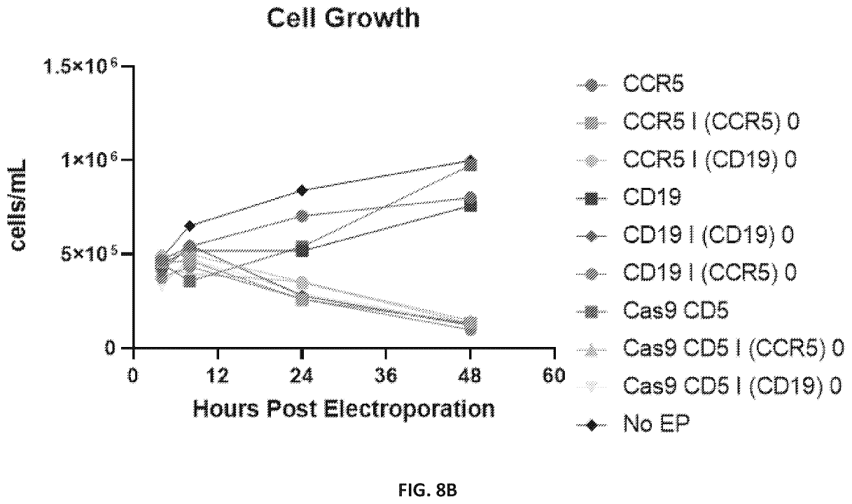

Cell viability and growth rate were assessed at times post-electroporation over the next 48 hours (FIGS. 8A-8B). FIGS. 9A and 9B show on-target editing as assessed by Next Generation Sequencing (NGS) analysis at the indicated time points following electroporation. For the cells that were co-electroporated with RNP complexes comprising Cpf1 nuclease, a gRNA directed to a target sequence and an inhibitor oligonucleotides directed to the gRNA (e.g., a gRNA directed to CCR5 and an inhibitor directed to the CCR5 gRNA or a gRNA directed to CD19 and an inhibitor directed to the CD19 gRNA), very little on-target editing, if any, was observed. However, for cells that were co-electroporated with RNP complexes comprising Cpf1 nuclease, a gRNA directed to a target sequence and inhibitor oligonucleotides directed to another gRNA (e.g., a gRNA directed to CCR5 and an inhibitor directed to a CD19 gRNA, or a gRNA directed to CD19 and an inhibitor directed to a CCR5 gRNA), on-target editing occurred to a comparable level as cells that did not receive the inhibitor oligonucleotides. The oligonucleotides specific for a different target had little to no effect on editing at the target sequence of the gRNA. These data indicate that the oligonucleotides described herein inhibit gene editing in a sequence-dependent manner.

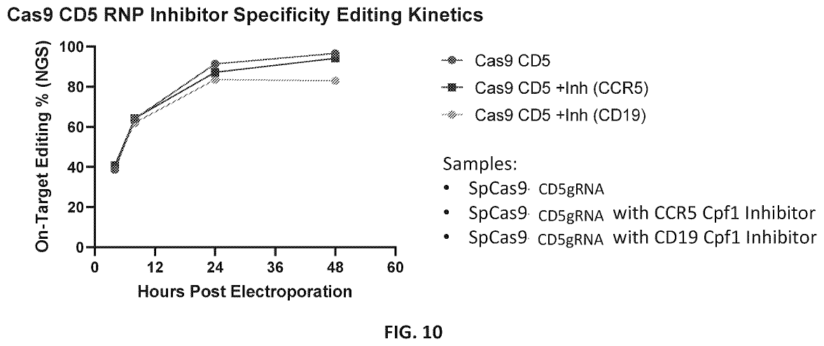

FIG. 10 shows on-target editing as assessed by Next Generation Sequencing (NGS) analysis at the indicated time points following electroporation RNPs comprising with SpCas9 nuclease and gRNAs targeting CD5 in the absence or presence of oligonucleotides specific to gRNAs targeting CCR5 or CD19. On-target editing occurred to a comparable level in cells that received the inhibitor oligonucleotides and cells that did not. These data indicate that oligonucleotides described herein did not significantly inhibit editing by SpCas9 nuclease.

### Example 4: Multiplex Editing Translocation Oligonucleotide Inhibition

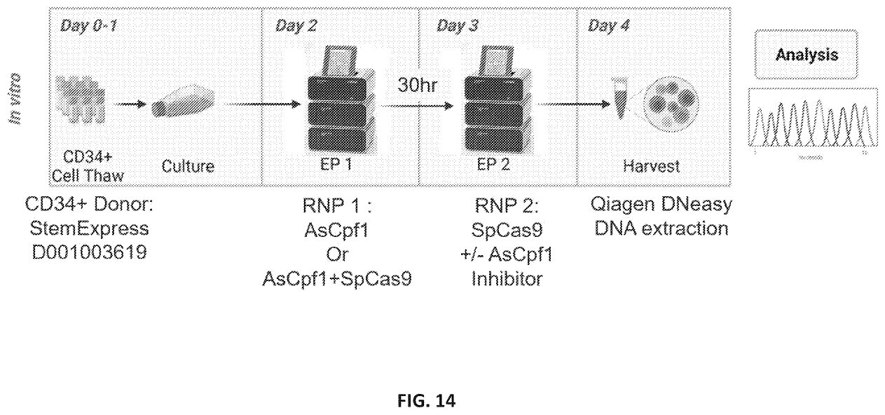

This example demonstrates that inhibition of genetic modification by the use of exemplary oligonucleotides of the disclosure is target-specific and significantly reduces the incidence of translocation events. An exemplary experimental workflow in shown in FIG. 14.

CD34+ cells were thawed as described in Examples 1 and 2. A group of cells were electroporated with ribonucleoprotein (RNP) complexes comprising Cpf1 nuclease from Acidaminococcus sp. BV3L6 (Alt-R Cas12a obtained from IDT) and a gRNA targeting a portion of the gene encoding CD19 comprising a targeting sequence provided by SEQ ID NO: 4 (see, Table B) or gRNA targeting a portion of the gene encoding CCR5 comprising a targeting sequence provided by SEQ ID NO: 3 (see, Table B) and simultaneously electroporated with RNP complexes comprising Cas9 nuclease from Streptococcus pyogenes and a gRNA targeting a portion of the gene encoding CD5 comprising a targeting sequence provided by SEQ ID NO: 5 (see, Table B).

A second group of cells was electroporated first with RNP complexes comprising Cpf1 nuclease and a gRNA targeting a portion of the gene encoding CD19 comprising a targeting sequence provided by SEQ ID NO: 4 (see, Table B) or gRNA targeting a portion of the gene encoding CCR5 comprising a targeting sequence provided by SEQ ID NO: 3 (see, Table B) followed by electroporation with RNP complexes comprising Cas9 nuclease from Streptococcus pyogenes and a gRNA targeting a portion of the gene encoding CD5 comprising a targeting sequence provided by SEQ ID NO: 5 (see, Table B).

Finally, a third group of cells was electroporated first with RNP complexes comprising Cpf1 nuclease and a gRNA targeting a portion of the gene encoding CD19 comprising a targeting sequence provided by SEQ ID NO: 4 (see, Table B) or a gRNA targeting a portion of the gene encoding CCR5 comprising a targeting sequence provided by SEQ ID NO: 3 (see, Table B) followed by electroporation with RNP complexes comprising Cas9 nuclease from Streptococcus pyogenes and a gRNA targeting a portion of the gene encoding CD5 comprising a targeting sequence provided by SEQ ID NO: 5 (see, Table B). This group was co-electroporated with inhibitor oligonucleotides targeting the Cpf1 gRNA sequence.

On-target editing and the presence to translocation products was assessed by Next Generation Sequencing (NGS) analysis following electroporation.

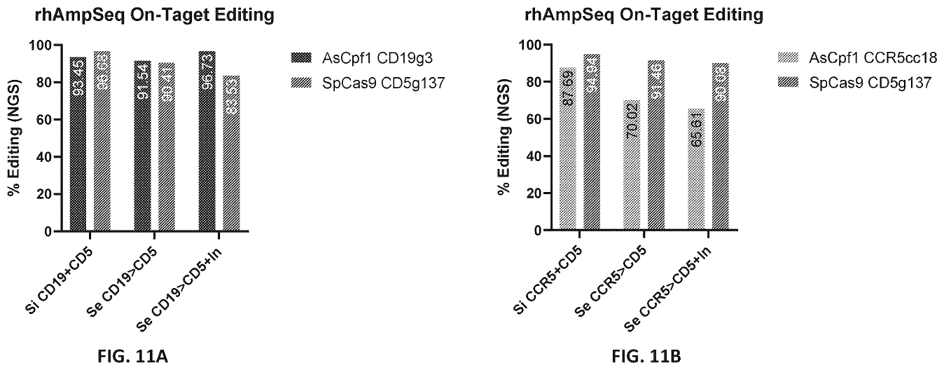

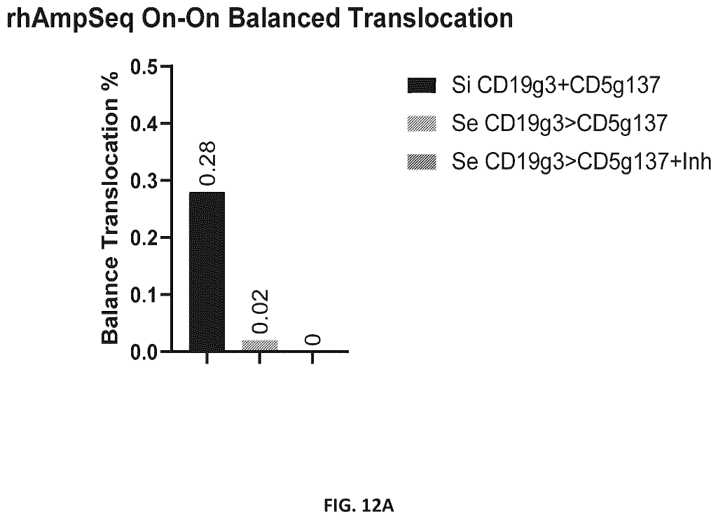

FIGS. 11A and 11B show on-target editing as assessed by Next Generation Sequencing (NGS) analysis of CD19 and CD5 or CCR5 and CD5, respectively, following simultaneous or sequential editing and in the absence or presence of oligonucleotides specific to the Cpf1 gRNAs. The on-target editing occurred to a comparable level in cells that received the inhibitor oligonucleotides and cells that did not. These data indicate that oligonucleotides that targeted Cpf1 gRNAs described herein did not significantly inhibit editing by SpCas9 nuclease.

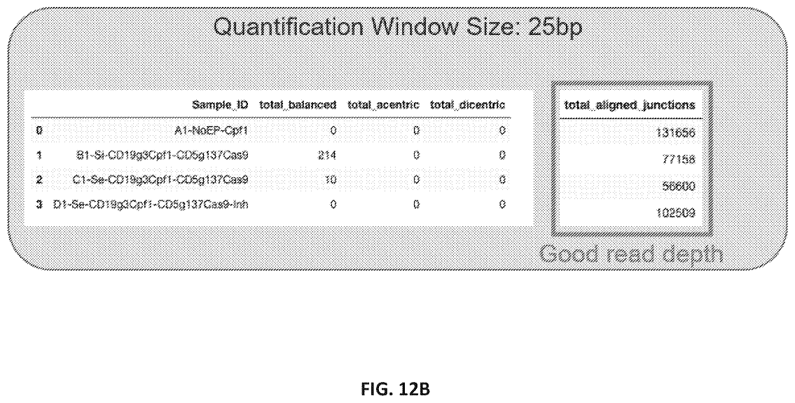

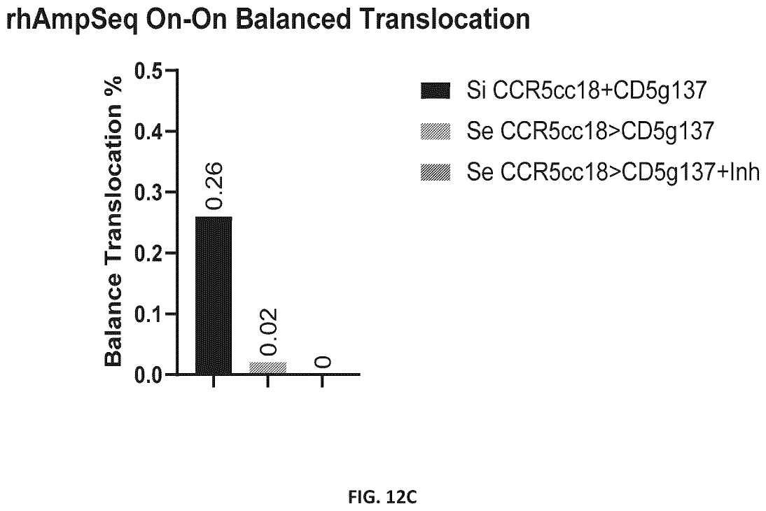

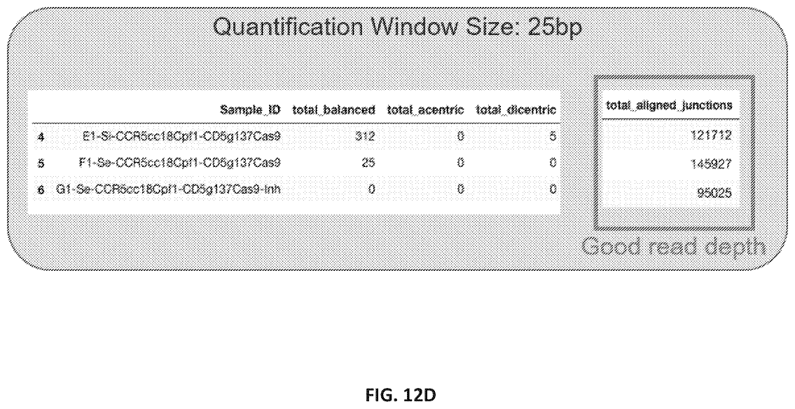

FIGS. 12A-12D show the percentage of balanced translocation products following editing of CD19 and CD5 or CCR5 and CD5. Sequential editing significantly reduced the percentage of translocation products, however the presence of oligonucleotides that targeted Cpf1 gRNAs reduced the percentage of translocation products to below detectable levels.

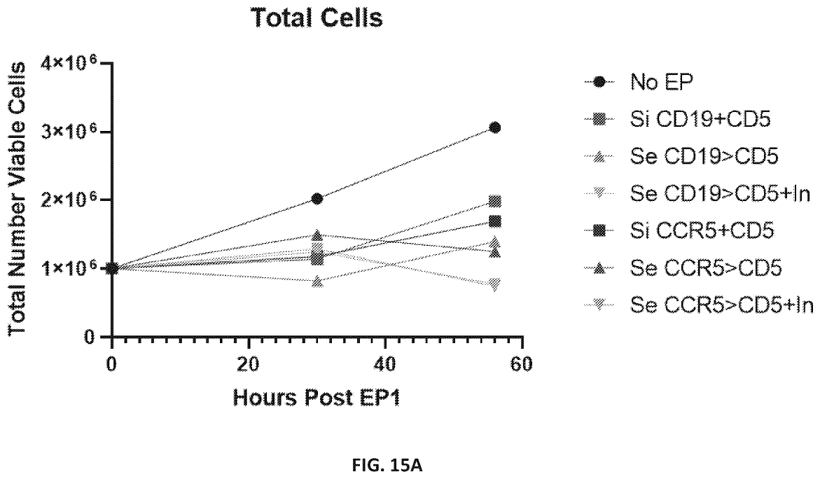

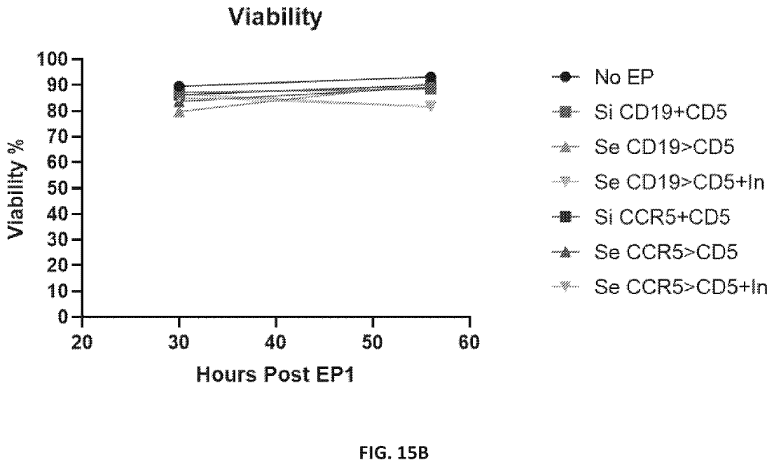

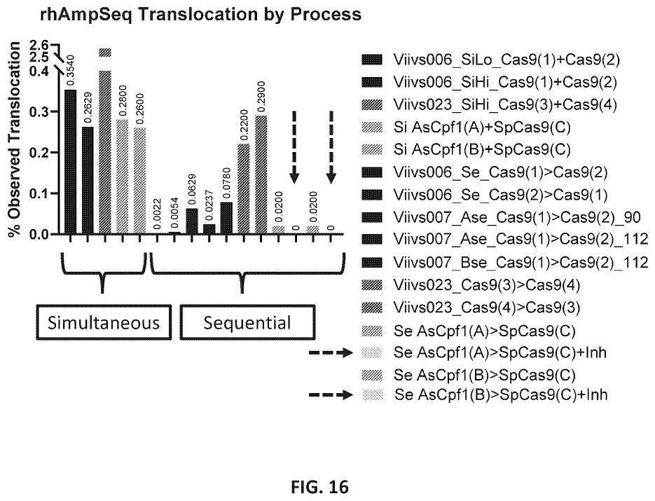

Following editing procedures, the effect of simultaneous and sequential electroporation with RNPs was assessed by cell viability and cell count analyses. Cell counts indicated that electroporation with inhibitors resulted in lower overall cell counts in edited cells. There was no effect on overall cell counts as a result of simultaneous editing compared with sequential editing (FIG. 15A). No effect on cell viability was observed as a result of any of the conditions tested (FIG. 15B).

These results indicate that the inhibitor oligonucleotides described herein do not significantly inhibit editing by other, non-targeted nucleases and also are effective in reducing the incidence of translocation products.

### Example 5: Molar Titration Ratios for Genome Editing

This example describes molar titration experiments for determining optimal dosage conditions for electroporation during genome editing.

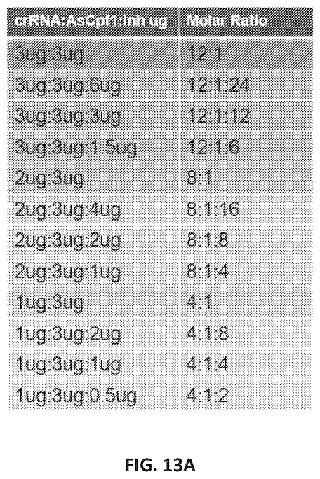

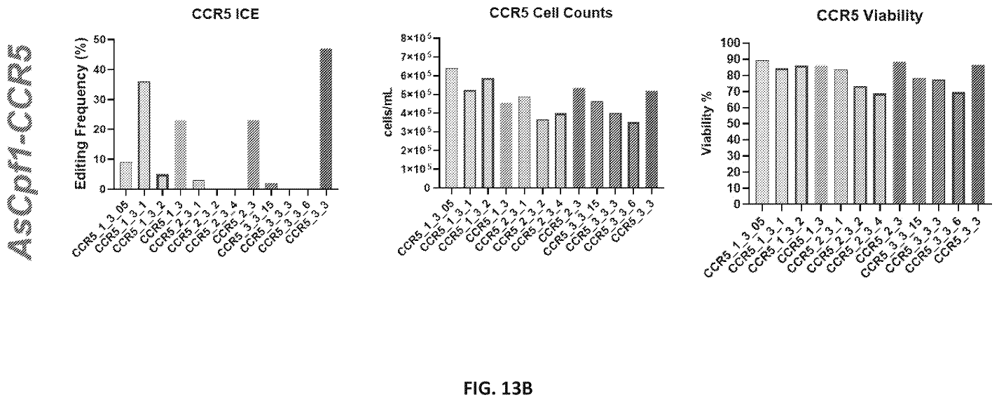

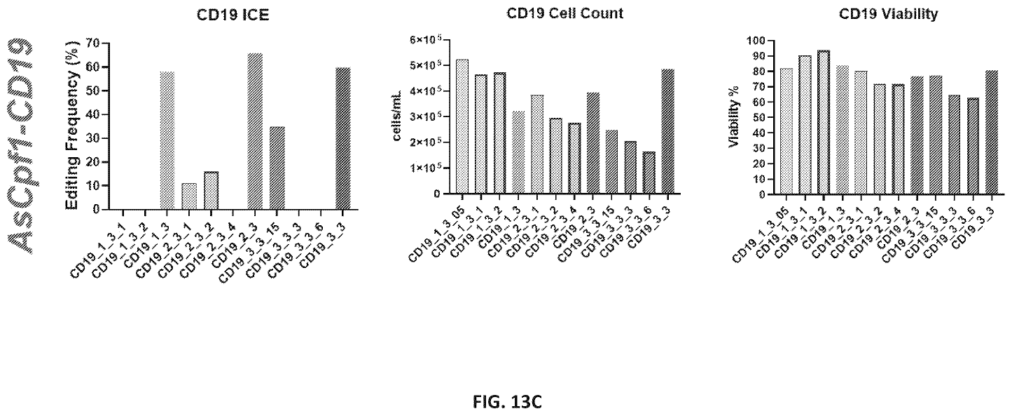

CD34+ cells were thawed and electroporated as previously described using the molar ratios shown in FIG. 13A. The results indicated that titrating the amount of oligonucleotide inhibitors rescued cell viability in CD34+ cell populations that were electroporated with RNPs targeting the CCR5 locus and CD34+ cell populations that were electroporated with RNPs targeting the CD19 locus (see FIGS. 13B and 13C), relative to initial experiments performed without dose titration (see FIGS. 2A-2B).

### Example 6: Multiplex Editing Using AsCpf1/SpCas9

This example describes a multiplex editing platform using a combination of AsCpf1 and SpCas9 for achieving high on-target editing in CD34+ cells.

As shown in FIG. 14, CD34+ donor cells were thawed, cultured, and on day 2, a population of cells were electroporated with a first RNP complex containing gRNAs targeting a target site (e.g., CCR5, CD19) and AsCpf1 or a combination of SpCas9 with AsCpf1. After 30 hours, cells were electroporated with a second RNP complex containing gRNAs targeting a target site (e.g., CCR5, CD19) and SpCas9 in the absence or in the presence of AsCpf1 inhibitor. Cells were harvested 24 hours following electroporation, DNA was extracted from the cells, and genomic editing was assessed by NGS analysis.

Following editing procedures, the effect of simultaneous and sequential electroporation with RNPs was assessed by cell viability and cell count analyses. Cell counts indicated that electroporation with inhibitors resulted in lower overall cell counts in edited cells. There was no effect on overall cell counts as a result of simultaneous editing compared with sequential editing (FIG. 15A). No effect on cell viability was observed as a result of any of the conditions tested (FIG. 15B).

FIG. 17 shows graphs of balanced genomic translocation (on-on translocation) determined by amplicon sequencing using the rhAmpSeq sequencing system. These results indicated that sequential editing procedures reduced balanced translocation 10-fold compared to simultaneous editing procedures. The results also confirmed that electroporation with inhibitors during editing procedures resulted in no balanced translocation.

### Example 7: In Vivo Applications of Inhibitor Oligonucleotides for Genome Editing

An example editing approach using the methods, cells, and agents described herein for genome editing is provided below.
1) CD34+ cells are thawed and cultured as previously described.
2) CD34+ cells are electroporated with a first RNP comprising AsCpf1 and gRNAs to a first target site as described above.
3) CD34+ cells are allowed to recover in vitro for 30 hours.
4) CD34+ cells are electroporated with a second RNP comprising SpCas9 and gRNAs to a second target site in addition to AsCpf1 inhibitor oligonucleotides.
5) A sample of the subpopulation of cells is harvested for downstream DNA extraction and analysis using NGS.
6) The remaining cells are engrafted into mouse models.
7) The engrafted cells are evaluated for characteristics to determine their ability to differentiate and the ability to engraft the patient and mediate graft-vs-tumor (GVT) effects.
8) Follow up with a cytotoxic agent, such as immune cells expressing a chimeric receptor (e.g., CAR T cell) or antibody-drug conjugate, wherein the epitope to which the cytotoxic agent binds is the same epitope that was modified and is no longer present on the donor engineered bone marrow graft. The targeted therapy should thus specifically target the lineage-specific cell-surface antigen, without simultaneously eliminating the bone marrow graft, in which the epitope is not present;
9) Administer a cytotoxic agent, such as immune cells expressing a chimeric receptor (e.g., CAR T cell) or antibody-drug conjugate that targets an epitope of a lineage specific cell-surface antigen. This targeted therapy would be expected to eliminate both cancerous cells as well as the patient's non-cancerous cells;
10) Pre-condition the AML patient using standard techniques, such as infusion of chemotherapy agents;
11) Administer the engineered donor bone marrow to the AML patient, allowing for successful engraftment.

The steps 8-10 result in the elimination of the patient's cancerous and normal cells expressing the targeted protein, while replenishing the normal cell population with donor cells that are resistant to the targeted therapy.

### Example 8: Treatment of Hematologic Disease

An example treatment regimen using the methods, cells, and agents described herein for acute myeloid leukemia is provided below.
1) Identify a patient with AML that is a candidate for receiving a hematopoietic cell transplant (HCT);
2) Identify a HCT donor with matched HLA haplotypes, using standard methods and techniques;
3) Extract the bone marrow from the donor;
4) Genetically manipulate the donor bone marrow cells ex vivo. Briefly, introduce a targeted modification (deletion, substitution) of a lineage-specific cell-surface antigen using a gRNA and a CRISPR/Cas nuclease (e.g., Cpf1), as described herein. Genetic editing is halted by introduction of a oligonucleotide that inhibits the specific gRNA. A further modification of the same or a different lineage-specific cell-surface antigen is introduced using a second gRNA targeting the same or a different antigen, and optionally, a second CRISPR/Cas nuclease.

Cells may be evaluated for characteristics to determine their ability to differentiate and the ability to engraft the patient and mediate graft-vs-tumor (GVT) effects.

### Optional Steps 5-7:

In some embodiments, Steps 5-7 provided below may be performed (once or multiple times) in an exemplary treatment method as described herein:
5) Pre-condition the AML patient using standard techniques, such as infusion of chemotherapy agents (e.g., etoposide, cyclophosphamide) and/or irradiation;
6) Administer the engineered donor bone marrow to the AML patient, allowing for successful engraftment;
7) Follow up with a cytotoxic agent, such as immune cells expressing a chimeric receptor (e.g., CAR T cell) or antibody-drug conjugate, wherein the epitope to which the cytotoxic agent binds is the same epitope that was modified and is no longer present on the donor engineered bone marrow graft. The targeted therapy should thus specifically target the lineage-specific cell-surface antigen, without simultaneously eliminating the bone marrow graft, in which the epitope is not present;

### Optional Steps 8-10:

In some embodiments, Steps 8-10 may be performed (once or multiple times) in an exemplary treatment method as described herein:
8) Administer a cytotoxic agent, such as immune cells expressing a chimeric receptor (e.g., CAR T cell) or antibody-drug conjugate that targets an epitope of a lineage specific cell-surface antigen. This targeted therapy would be expected to eliminate both cancerous cells as well as the patient's non-cancerous cells;
9) Pre-condition the AML patient using standard techniques, such as infusion of chemotherapy agents;
10) Administer the engineered donor bone marrow to the AML patient, allowing for successful engraftment.

The steps 8-10 result in the elimination of the patient's cancerous and normal cells expressing the targeted protein, while replenishing the normal cell population with donor cells that are resistant to the targeted therapy.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. An oligonucleotide, comprising a first region that is complementary to a targeting domain of a gRNA and a second region that is complementary to a CRISPR RNA (crRNA) sequence for a CRISPR/Cas nuclease, wherein the oligonucleotide does not occur naturally,
wherein the oligonucleotide reduces genomic editing at a target sequence complementary to the targeting domain of the gRNA; and
wherein the first region comprises at least 10 nucleotides and the second region comprises at least 10 nucleotides.

2. The oligonucleotide of claim 1, wherein the CRISPR/Cas nuclease is Cpf1.

3. The oligonucleotide of claim 1, wherein the CRISPR/Cas nuclease is MAD7™.

4. The oligonucleotide of any one of claims 1-3, wherein the targeting domain is complementary to a eukaryotic gene.

5. The oligonucleotide of any one of claims 1-4, wherein the oligonucleotide binds to the targeting domain and/or the crRNA sequence and reduces interaction between the targeting domain and/or crRNA sequence and the CRISPR/Cas nuclease.

6. The oligonucleotide of any one of claims 1-5, wherein the oligonucleotide reduces interaction between the gRNA and the CRISPR/Cas nuclease.

7. The oligonucleotide of any one of claims 1-6, wherein the oligonucleotide inhibits formation or maintenance of a ribonucleoprotein (RNP) complex comprising the gRNA and the CRISPR/Cas nuclease.

8. The oligonucleotide of any one of claims 1-7, wherein the oligonucleotide inhibits nuclease activity of a RNP complex comprising the gRNA and the CRISPR/Cas nuclease and/or reduces interaction between the RNP complex and the target sequence in the genome of a cell.

9. The oligonucleotide of any one of claims 1-8, wherein the first region comprises at least 11, 12, 13, 14, 15, 15, 16, 17, 19, 20, 21, 22, 23, 24, 25, 26, 27, 28, 29, 30, 31, 32, 33, 34, 35, 36, 37, 38, 39, 40, 41, 42, 43, 44, or 45 nucleotides and the second region comprises at least 11, 12, 13, 14, 15, 15, 16, 17, 19, 20, 21, 22, 23, 24, 25, 26, 27, 28, 29, 30, 31, 32, 33, 34, 35, 36, 37, 38, 39, 40, 41, 42, 43, 44, or 45 nucleotides.

10. The oligonucleotide of any one of claims 1-9, wherein the oligonucleotide comprises one or more nucleotides that comprise a chemical modification.

11. The oligonucleotide of any one of claims 1-10, wherein at least 1, 2, 3, 4, 5, 6, 7, 8, 9, 10, 15, 20, 25, 30, 35, or 40 nucleotides of the oligonucleotide comprise a chemical modification.

12. The oligonucleotide of any one of claims 1-11, wherein at least 10, 20, 50, 75, or 100% of the nucleotides of the oligonucleotide comprise a chemical modification.

13. The oligonucleotide of any one of claims 10-12, wherein the chemical modification is a phosphorothioate linkage.

14. The oligonucleotide of claim 13, wherein each nucleotide of the oligonucleotide comprises a phosphorothioate linkage.

15. The oligonucleotide of any one of claims 1-14, wherein the oligonucleotide is 10-100 nucleotides in length.

16. The oligonucleotide of claim 15, wherein the oligonucleotide is 38, 39, 40, 41, 42, 43, 44, or 45 nucleotides in length.

17. The oligonucleotide of any one of claims 1-16, wherein the oligonucleotide comprises a sequence selected from the group consisting of SEQ ID NOs: 1 and 2.

18. The oligonucleotide of any one of claims 1-17, wherein the second region of the oligonucleotide comprises a sequence of SEQ ID NO: 13.

19. The oligonucleotide of any one of claims 2-18, wherein the Cpf1 nuclease is derived from Provetella spp. or Francisella spp., Acidaminococcus sp. (AsCpf1), Lachnospiraceae bacterium (LbCpf1), or Eubacterium rectale.

20. The oligonucleotide of any one of claims 1-19, wherein the Cpf1 nuclease comprises an amino acid sequence with at least 80, 85, 90, 95, 99, or 100% identity to an amino acid sequence selected from the group consisting of SEQ ID NOs: 12, 14, and 15.

21. A method of producing a genetically engineered cell, comprising:
(a) contacting a cell with
(i) a first guide RNA (gRNA) and
(ii) a CRISPR/Cas nuclease that binds the first gRNA, thus forming a ribonucleoprotein (RNP) complex under conditions suitable for the first gRNA of (i) to form and/or maintain an RNP complex with the CRISPR/Cas nuclease of (ii) and for the RNP complex to bind a first target sequence in the genome of the cell, and
(b) contacting the cell with an oligonucleotide, wherein the oligonucleotide reduces genomic editing at the first target sequence.

22. The method of claim 21, wherein the CRISPR/Cas nuclease is Cpf1.

23. The method of claim 21, wherein the CRISPR/Cas nuclease is MAD7™.

24. The method of any one of claims 21-23, wherein the oligonucleotide comprises a first region that is complementary to a targeting domain of the first gRNA or a portion thereof, and a second region that is complementary to a CRISPR RNA (crRNA) sequence in the first gRNA or a portion thereof.

25. The method of any one of claims 21-24, wherein the oligonucleotide is an oligonucleotide of any of claims 1-20.

26. The method claim 24 or 25, wherein the targeting domain of the target gRNA is capable of binding a first target sequence and a CRISPR RNA (crRNA) sequence for the CRISPR/Cas nuclease.

27. The method of any one of claims 24-26, wherein the targeting domain corresponds to the first target sequence adjacent to a protospacer-adjacent motif (PAM) in a genome of the cell.

28. The method of any one of claims 21-27, wherein the contacting of (b) occurs simultaneously or in temporal proximity with the contacting of (a).

29. The method of any one of claims 21-28, wherein the contacting of (b) occurs within 1, 2, 3, 4, 5, 6, 7, 8, 9, 10, 11, 12, 18, or 24 hours after the contacting of (a).

30. The method of any of claims 21-29, further comprising:
(c) contacting the cell with (iii) a second gRNA comprising a second targeting domain capable of binding a second target sequence and a crRNA sequence for a CRISPR/Cas nuclease;
wherein the second targeting domain and second target sequence are different than the targeting domain of the first gRNA and the first target sequence.

31. The method of claim 30, wherein (c) further comprises contacting the cell with (iv) a CRISPR/Cas nuclease that binds the second gRNA, thus forming a ribonucleoprotein (RNP) complex under conditions suitable for the second gRNA of (iii) to form and/or maintain an RNP complex with the CRISPR/Cas nuclease of (iv) and for the RNP complex to bind a second target sequence in the genome of the cell.

32. The method of claim 31, wherein the CRISPR/Cas nuclease of (iv) does not comprise a Cpf1 nuclease.

33. The method of claim 31 or 32, wherein the CRISPR/Cas nuclease of (iv) is a Cas9 nuclease.

34. The method of claim 31, wherein the CRISPR/Cas nuclease of (iv) comprises a Cpf1 nuclease.

35. The method of any one of claims 30-34, wherein the contacting of (c) occurs simultaneously or in temporal proximity with the contacting of (b).

36. The method of any one of claims 30-35, wherein the contacting of (c) occurs within 1, 2, 3, 4, 5, 6, 7, 8, 9, 10, 11, 12, 18, or 24 hours after the contacting of (b).

37. The method of any one of claims 30-36, further comprising:
(d) contacting the cell with a second oligonucleotide, wherein the second oligonucleotide reduces genomic editing at the second target sequence.

38. The method of claim 37, wherein the contacting of (d) and the contacting of (c) occur simultaneously or in temporal proximity to one another.

39. The method of claim 37 or 38, wherein the contacting of (d) occurs within 1, 2, 3, 4, 5, 6, 7, 8, 9, 10, 11, 12, 18, or 24 hours after the contacting of (c).

40. The method of any one of claims 37-39, wherein the second oligonucleotide is an oligonucleotide of any of one claims 1-20.

41. The method of any one of claims 30-40, wherein the oligonucleotide of (b) does not substantially bind the second gRNA of (iii) and/or a RNP complex of the second gRNA of (iii) and the CRISPR/Cas nuclease of (iv).

42. The method of any one of claims 30-41, wherein the oligonucleotide of (b) does not substantially inhibit formation or maintenance of the RNP complex comprising the second gRNA of (iii) and the CRISPR/Cas nuclease of (ii) or the CRISPR/Cas nuclease of (iv).

43. The method of any one of claims 30-42, wherein the oligonucleotide of (b) does not substantially inhibit the ability of the RNP complex comprising the second gRNA of (iii) and the CRISPR/Cas nuclease of (ii) or the CRISPR/Cas nuclease of (iv) to bind the second target sequence in the genome of the cell.

44. The method of any one of claims 30-43, wherein the oligonucleotide of (b) does not substantially inhibit nuclease activity of the RNP complex comprising the second gRNA of (iii) and the CRISPR/Cas nuclease of (ii) or the CRISPR/Cas nuclease of (iv) to bind the second target sequence in the genome of the cell.

45. The method of any one of claims 37-44, wherein the second oligonucleotide of (d) does not substantially bind the gRNA of (i) and/or the RNP complex comprising the first gRNA of (i) and the CRISPR/Cas nuclease of (ii).

46. The method of any one of claims 37-45, wherein the second oligonucleotide of (d) does not substantially inhibit formation or maintenance of the RNP complex comprising the first gRNA of (i) and the CRISPR/Cas nuclease of (ii).

47. The method of any one of claims 37-46, wherein the second oligonucleotide of (d) does not substantially inhibit the ability of the RNP complex comprising the first gRNA of (i) and the CRISPR/Cas nuclease of (ii) to bind the first target sequence in the genome.

48. The method of any one of claims 37-47, wherein the second oligonucleotide of (d) does not substantially inhibit nuclease activity of the RNP complex comprising the first gRNA of (i) and the CRISPR/Cas nuclease of (ii) to bind the first target sequence in the genome of the cell.

49. The method of any of claims 21-48, wherein the cell is a hematopoietic cell.

50. The method of claim 49, wherein the hematopoietic cell is a hematopoietic stem cell.

51. The method of claim 49 or 50, wherein the cell is a hematopoietic progenitor cell.

52. The method of any one of claims 21-48, wherein the cell is an immune effector cell.

53. The method of any one of claims 21-48 or 52, wherein the cell is a lymphocyte.

54. The method of any one of claims 21-48, 52, or 53, wherein the cell is a T-lymphocyte.

55. The method of claim 52 or 53, wherein the cell is a natural killer (NK) cell.

56. The method of any of claims 21-48, wherein the cell is a stem cell.

57. The method of claim 56, wherein the stem cell is selected from the group consisting of: an embryonic stem cell (ESC), an induced pluripotent stem cell (iPSC), a mesenchymal stem cell, or a tissue-specific stem cell.

58. The method of any one of claims 21-57, wherein the contacting of (a) comprises introducing (i) and (ii) into the cell in the form of a pre-formed ribonucleoprotein (RNP) complex.

59. The method of any one of claims 30-58, wherein the contacting of (c) comprises introducing the second gRNA of (iii) and the CRISPR/Cas nuclease of (iv) into the cell in the form of a pre-formed ribonucleoprotein (RNP) complex.

60. The method of any one of claims 21-57, wherein the contacting of (a) comprises introducing (i) and/or (ii) into the cell in the form of a nucleic acid encoding the first gRNA of (i), and/or the CRISPR/Cas nuclease of (ii); and/or the contacting of (b) comprises introducing the oligonucleotide into the cell in the form of a nucleic acid encoding the oligonucleotide.

61. The method of claim 60, wherein the nucleic acid encoding the first gRNA of (i), and/or the CRISPR/Cas nuclease of (ii), and/or the oligonucleotide is an RNA, preferably an mRNA or an mRNA analog.

62. The method of any one of claims 30-61, wherein the contacting of (c) comprises introducing the second gRNA of (iii) and/or the CRISPR/Cas nuclease of (iv) into the cell in the form of a nucleic acid encoding the second gRNA of (iii) and/or the second CRISPR/Cas nuclease of (iv); and/or the contacting of (d) comprises introducing the second oligonucleotide into the cell in the form of a nucleic acid encoding the second oligonucleotide.

63. The method of claim 62, wherein the nucleic acid encoding the second gRNA of (iii), the second CRISPR/Cas nuclease of (iv), and/or the second oligonucleotide is an RNA, preferably an mRNA or an mRNA analog.

64. The method of any one of claims 58-63, wherein the RNP complex is introduced into the cell via electroporation.

65. A genetically engineered cell, or descendent thereof, produced by a method of any of claims 21-64.

66. A cell population, comprising the genetically engineered cell, or a descendant thereof, of claim 65.

67. A pharmaceutical composition comprising the cell, or a descendant thereof, of claim 65 or the cell population of claim 66.

68. A ribonucleoprotein particle (RNP) comprising:
a CRISPR/Cas nuclease,
a first gRNA, and
the oligonucleotide of any one of claims 1-20.

69. The ribonucleoprotein particle (RNP) of claim 68, wherein the CRISPR/Cas nuclease is Cpf1.

70. The ribonucleoprotein particle (RNP) of claim 68, wherein the CRISPR/Cas nuclease is MAD7™.

71. A system comprising:
a CRISPR/Cas nuclease,
a first gRNA,
the oligonucleotide of any of claims 1-20, and
a second gRNA.

72. The system of claim 71, wherein the CRISPR/Cas nuclease is Cpf1.

73. The system of claim 61, wherein the CRISPR/Cas nuclease is MAD7™.

74. The system of any one of claims 71-73, further comprising a second CRISPR/Cas nuclease.

75. A method, comprising administering to a subject in need thereof the cell, or descendant thereof, of claim 65, the cell population of claim 66, or the pharmaceutical composition of claim 67.

76. The method of claim 75, wherein the cell or descendant thereof or the cells of the cell population comprise a modification in a first gene relative to a wild-type counterpart cell.

77. The method of claim 75 or 76, wherein the cell or descendant thereof or the cells of the cell population comprise a modification to a second gene relative to a wild-type cell of the same type.

78. The method of any of claims 75-77, further comprising administering to the subject a therapeutically effective amount of at least one agent that targets a product encoded by the first gene or a wildtype copy thereof, wherein the agent comprises an antigen binding fragment that binds the product encoded by the first gene or a wildtype copy thereof.

79. The method of claim 78, wherein administration of the at least one agent targeting the product encoded by the first gene or a wildtype copy thereof occurs simultaneously or in temporal proximity with administration of the cell, or descendant thereof, of claim 65, the cell population of claim 66, or the pharmaceutical composition of claim 67.

80. The method of claim 78 or 79, wherein administration of the at least one agent targeting the product encoded by the first gene or a wildtype copy thereof occurs after administration of the cell, or descendant thereof, of claim 65, the cell population of claim 66, or the pharmaceutical composition of claim 67.

81. The method of claim 78 or 79, wherein administration of the at least one agent targeting the product encoded by the first gene or a wildtype copy thereof occurs before administration of the cell, or descendant thereof, of claim 65, the cell population of claim 66, or the pharmaceutical composition of claim 67.

82. The method of any one of claims 75-81, further comprising administering to the subject a therapeutically effective amount of at least one agent that targets a product encoded by the second gene or a wildtype copy thereof, wherein the agent comprises an antigen binding fragment that binds the product encoded by the second gene or a wildtype copy thereof.

83. The method of claim 82, wherein administration of the at least one agent targeting the product encoded by the second gene or a wildtype copy thereof occurs simultaneously or in temporal proximity with administration of the cell, or descendant thereof, of claim 65, the cell population of claim 66, or the pharmaceutical composition of claim 67.

84. The method of claim 83, wherein administration of the at least one agent targeting the product encoded by the second gene or a wildtype copy thereof occurs after administration of the cell, or descendant thereof, of claim 65, the cell population of claim 66, or the pharmaceutical composition of claim 67.

85. The method of claim 83, wherein administration of the at least one agent targeting the product encoded by the second gene or a wildtype copy thereof occurs before administration of the cell, or descendant thereof, of claim 65, the cell population of claim 66, or the pharmaceutical composition of claim 67.

86. The method of any one of claims 83-85, wherein administration of the at least one agent targeting the product encoded by the second gene or a wildtype copy thereof occurs simultaneously or in temporal proximity with administration of the at least one agent targeting the product encoded by the first gene or a wildtype copy thereof.

87. The method of any of claims 83-86, wherein administration of the at least one agent targeting the product encoded by the second gene or a wildtype copy thereof occurs after administration of the at least one agent targeting the product encoded by the first gene or a wildtype copy thereof.

88. The method of any one of claims 83-86, wherein administration of the at least one agent targeting the product encoded by the second gene or a wildtype copy thereof occurs before administration of the at least one agent targeting the product encoded by the first gene or a wildtype copy thereof.

89. The method of any one of claims 78-88, wherein the agent that targets the product encoded by the first gene or a wildtype copy thereof and/or the agent that targets the product encoded by the second gene or a wildtype copy thereof is a cytotoxic agent.

90. The method of claim 89, wherein the cytotoxic agent is an antibody-drug conjugate or an immune effector cell expressing a chimeric antigen receptor (CAR).

91. The method of any one of claims 75-90, wherein the subject has a disease associated with cells expressing the modified gene or a wildtype copy thereof.

92. The method of any one of claims 75-91, wherein the subject has a cancer associated with cancer stem cells.

93. The method of any one of claims 75-92, wherein the subject has a hematopoietic malignancy.

94. The method of any one of claims 75-91, wherein the subject has an autoimmune disease.


SEQUENCE LISTING (15 sequences)

SEQ ID NO: 1 - DNA, 41 bp, synthetic construct
  source 1..41: mol_type = other DNA
  modified_base 1..41: mod_base = OTHER; note = Phosphorothioate linkage
tgctggtcatcctcatcctgaatctacaagagtagaaatta

SEQ ID NO: 2 - DNA, 41 bp, synthetic construct
  source 1..41: mol_type = other DNA
  modified_base 1..41: mod_base = OTHER; note = Phosphorothioate linkage
ctggtctcgggagtccccgctatctacaagagtagaaatta

SEQ ID NO: 3 - DNA, 21 bp, unidentified
  source 1..21: mol_type = genomic DNA; note = Acidaminococcus sp.
tcaggatgaggatgaccagca

SEQ ID NO: 4 - DNA, 21 bp, unidentified
  source 1..21: mol_type = genomic DNA; note = Acidaminococcus sp.
agcggggactcccgagaccag

SEQ ID NO: 5 - DNA, 20 bp, Streptococcus pyogenes
  source 1..20: mol_type = genomic DNA
catagctgatggtacccccc

SEQ ID NO: 6 - DNA, 20 bp, Streptococcus pyogenes
  source 1..20: mol_type = genomic DNA
ggtgggggcagctgacaacc

SEQ ID NO: 7 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 24 aa, synthetic construct
IYIWAPLAGTCGVLLLSLVITLYC

SEQ ID NO: 9 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 10 - protein, 41 aa, synthetic construct
KRGRKKLLYIFKQPFMRVQTTQEEDGCSCRFPEEEEGGCEL

SEQ ID NO: 11 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 1307 aa, unidentified
  source 1..1307: note = Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 13 - DNA, 20 bp, synthetic construct
  source 1..20: mol_type = other DNA
atctacaagagtagaaatta

SEQ ID NO: 14 - protein, 1325 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLKRPAATKKAGQAKKKKGSYPYDVPDYAYPYDVPDYAYPYDVPDYAGSPKKKRKVGSHHHHHH

SEQ ID NO: 15 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL